(19) United States
(12) Patent Application Publication
(10) Pub. No.: US 2008/0063701 A1
(43) Pub. Date: Mar. 13, 2008

(54) Vector

(76) Inventors: Michael Keller, London (GB); Michael Jorgensen, London (GB); Andrew David Miller, London (GB); Eric Perouzel, London (GB)

Correspondence Address: FAY SHARPE LLP, 1100 SUPERIOR AVENUE, SEVENTH FLOOR, CLEVELAND, OH, 44114, US

(21) Appl. No.: 11/172,641

(22) Filed: Jul. 1, 2005

Related U.S. Application Data
(60) Provisional application No. 60/614,085, filed on Sep. 9, 2004.

(30) Foreign Application Priority Data
Aug. 13, 2004 (GB) GB 0418172.3

(51) Int. Cl. A61K 31/7105; A61K 9/127; A61P 43/00; C12N 5/06

(52) U.S. Cl. 424/450; 428/402.2; 435/375; 514/44

(57) ABSTRACT

The present invention relates to a non-viral delivery vector comprising a liposome, wherein one or more lipids of the liposome are coupled, reversibly or irreversibly, to one or more polymers, and wherein the liposome comprises siRNA.

### FIELD OF INVENTION

The present invention relates to a non-viral delivery vector comprising siRNA. The present invention also relates to a targeted non-viral delivery vector, methods of preparing the vectors, methods of using the vectors and uses thereof.

### BACKGROUND TO THE INVENTION

Methods for viral DNA delivery suffer from many problems including immune responses, inability to deliver viral DNA vectors repeatedly, difficulty in generating high viral titres, and the possibility of infectious virus. Non-viral delivery methods provide an alternative system that is devoid of these problems and has therefore prompted the development of less hazardous, non-viral approaches to gene transfer.

A non-viral transfer system of great potential involves the use of cationic liposomes, which usually consist of a neutral phospholipid and a cationic lipid. They have been used to transfer DNA, mRNA, antisense oligonucleotides, proteins, and drugs into cells. A number of cationic liposomes are commercially available and many new cationic lipids have recently been synthesised. The efficacy of these liposomes has been illustrated by both in vitro and in vivo.

RNAi in animals and basal eukaryotes, quelling in fungi, and post-transcriptional gene silencing in plants are examples of a broad family of phenomena collectively called RNA silencing (12,13). The phenomenon of specific RNA inactivation was first discovered in plants as a defence mechanism against virus infection (14), and later in C. elegans (15,16). The common features of RNA silencing are the production of small (21-23 nt) double stranded RNAs called siRNA that act as specific determinants for down-regulation of gene expression (13). The key enzyme in the intracellular production of small double stranded RNAs is Dicer, a cytosolic ribonuclease III that digests long double stranded RNA into 21-23nt units (13,17,18). These short double stranded RNAs are unwound, and one of the two strands becomes associated with a complex of proteins and the target transcript-designated as the RNA-induced silencing complex (RISC)—that leads to target RNA destruction (19). The discovery that synthetic double stranded RNA sequences (siRNA) of 21-23 nucleotides can surrogate in this process and have the potential to specifically down-regulate gene function in cultured mammalian cells (20), has opened the gateway to applications of the RNAi concept in functional genomics programs and even in therapy. Early research in vivo has demonstrated the potential of synthetic siRNA and transgenic siRNA to down-regulate both exogenous and endogenous gene expression in adult mice (21,22). Thus potential side effects caused by siRNA appear to be the stimulation of the interferon system but little more (23,24).

Whilst the delivery of many different nucleic acids has been extensively described, research into the delivery of siRNA is at a preliminary level. Thus, in spite of the widespread use of cationic lipid/liposome systems to deliver plasmid DNA (pDNA) and oligodeoxynucleotides (ODNs) to cells (7,9,25-28), there has been little reported in the literature concerning the formulation of siRNA with cationic lipid/liposomes and its delivery to cells (siFection) either in vitro or in vivo. Even basic studies concerning the formulation of cationic lipid/liposome systems with siRNA are yet to be reported.

The present invention seeks to provide improvements in the non-viral delivery of siRNA.

### SUMMARY OF THE INVENTION

One reason why research into the delivery of siRNA is still at a preliminary level may be the apparent confusion that all nucleic acids are much alike and should be delivered to cells in comparable ways using comparable delivery systems. For instance, due to the similar chemical nature of oligonucleotides (ODN), pDNA and siRNA, one would expect that the formulation of siRNA and ODN/pDNA would behave similarly, and that mechanistically, the two species display similar features.

Superficially this is true. Both pDNA and siRNA have anionic phosphodiester backbones with identical negative charge/nucleotide (nt) ratios and should therefore interact electrostatically with cationic liposome/lipid-systems to form cationic lipid-nucleic acid (lipoplex) particles able to transfer the nucleic acids into cells. However, pDNA and siRNA are otherwise very different in molecular weight and molecular topography from each other with potentially important consequences.

All pDNA condenses into small nanoparticles of 60-100 nm subsequent to neutralization of 70-90% of its phosphodiester backbone charge with a cationic agent (27-31). Cationic agent condensed pDNA can then exist in a variety of different morphologies depending upon the cationic condensing agent, such as spherical, toroids and rods (27,28). Irrespective of the agent, there is a minimal size for pDNA condensation corresponding to around 400 nucleotides (32). Such behaviour ensures that pDNA is almost entirely encapsulated or encased by the cationic agent and protected from enzymatic or physical degradation within nanometric particles (26,33-40).

In contrast to pDNA, siRNA cannot condense into particles of nanometric dimensions being already a small sub-nanometric nucleic acid. Therefore, electrostatic interactions between siRNA and a cationic lipid/liposome system pose two potential problems. Firstly, a relatively uncontrolled interaction process leading to siRNA-lipoplex (LsiR) particles of excessive size and poor stability. Secondly, incomplete encapsulation of siRNA molecules thereby exposing siRNA to potential enzymatic or physical degradation prior to delivery to cells.

Such considerations make it clear that pDNA and siRNA are completely different kinds of nucleic acids.

Accordingly, the present invention is based in part, upon the surprising finding that a non-viral delivery vector comprising siRNA and a liposome coupled to a polymer dramatically stabilises the liposomes against aggregation without impairing the power of the siRNA to downregulate the target gene. In particular, liposomes comprising siRNA can be generated with an average size of 30-60 nm, which can be incubated with varying amounts of polymer which can form a bond—such as a covalent bond—between the polymer (eg. functional groups of the polymer) and the lipid.

Due to the similar chemical nature of oligonucleotides (ODN), pDNA and siRNA, one would expect that the formulation of siRNA and ODN/pDNA would behave similarly, and that mechanistically, the two species would display similar features. In other words, it would have been expected that PEGylated siRNA complexes, even at a low degree of pegylation, would not mediate the down-regulation of a target gene, since the literature described herein indicates that these complexes either fail to be internalised, fail to escape from endosomes, or fail to find the target compartment/molecule necessary to mediate its biological action.

Evidence presented herein surprisingly indicates that such assumptions do not apply to siRNA.

The present invention is also based in part upon the surprising finding that the non-viral vectors described herein can be further incubated with an agent (eg. a targeting moiety—such as an oxidised IgG antibody). Without wishing to be bound by any particular theory, it appears that rather than the polymer covering the excess free functional groups of the lipid exposed on the surface of the liposome, a second layer of a ligand—such as a targeting moiety on the non-viral vector is formed.

### SUMMARY ASPECTS OF THE PRESENT INVENTION

In a first aspect, the present invention relates to a non-viral delivery vector comprising a liposome, wherein one or more lipids of the liposome are coupled, reversibly or irreversibly, to one or more polymers, and wherein the liposome comprises siRNA.

In a second aspect, the present invention relates to a targeted non-viral delivery vector comprising a liposome, wherein one or more lipids of the liposome are coupled, reversibly or irreversibly, to one or more polymers and one or more agents, and wherein the liposome comprises siRNA.

In a third aspect, the present invention relates to a method for delivering siRNA to a cell, comprising the step of providing to the environment of a cell, tissue or organ the non-viral delivery vector according to the first aspect of the present invention or the targeted delivery vector according to the second aspect of the present invention.

In a fourth aspect, the present invention relates to a non-viral delivery vector according to the first aspect of the present invention or the targeted delivery vector according to the second aspect of the present invention for use in the delivery of siRNA to a cell, tissue or organ.

In a fifth aspect, the present invention relates to the use of a non-viral delivery vector according to the first aspect of the present invention or the targeted delivery vector according to the second aspect of the present invention in the manufacture of a composition for the delivery of siRNA to a cell, tissue or organ.

In a sixth aspect, the present invention relates to a process for preparing a non-viral delivery vector comprising a liposome, wherein one or more lipids of the liposome are coupled, reversibly or irreversibly, to one or more polymers, and wherein the liposome comprises siRNA, comprising the steps of: (i) contacting the siRNA with a liposome; and (ii) coupling, reversibly or irreversibly, the liposome formed in step (i) to the polymer(s).

In a seventh aspect, the present invention relates to a process for preparing a targeted non-viral delivery vector comprising a liposome, wherein one or more lipids of the liposome are coupled, reversibly or irreversibly, to one or more polymers and one or more agents, and wherein the liposome comprises siRNA, comprising the steps of: (i) contacting the siRNA with the liposome; (ii) coupling, reversibly or irreversibly, the liposome formed in step (i) to a polymer(s); and (iii) coupling, reversibly or irreversibly, the liposome formed in step (i) or step (ii) with one or more agent(s).

In a eighth aspect, the present invention relates to a method comprising the steps of: (i) providing a vector according to the first or second aspects of the present invention; (ii) optionally contacting the vector with a cryo-protectant; and (iii) freeze-drying the vector.

In a ninth aspect, the present invention relates to a freeze-dried vector obtainable or obtained by the method according to the eight aspect of the present invention.

In a tenth aspect, the present invention relates to a liposome comprising a lipid and a coupling moiety wherein the distance between the lipid and the coupling moiety is at least 1.5 nm.

In a eleventh aspect, the present invention relates to a pharmaceutical composition comprising the non-viral delivery vector according to the first aspect of the present invention or the targeted delivery vector according to the second aspect of the present invention or the liposome according to the tenth aspect and a pharmaceutically acceptable carrier or diluent.

In an twelfth aspect, the present invention relates to a method of treating a disease in a subject comprising administering to said subject a medically effective amount of a non-viral delivery vector according to the first aspect of the present invention or a targeted delivery according to the second aspect of the present invention, a liposome according to the tenth aspect or a pharmaceutical composition according to the eleventh aspect of the present invention.

In a thirteenth aspect, the present invention relates to a non-viral delivery vector according to the first aspect of the present invention or the targeted delivery vector according to the second aspect of the present invention or a liposome according to the tenth aspect for use in the treatment of a disease.

In a fourteenth aspect, the present invention relates to the use of a non-viral delivery vector according to the first aspect of the present invention or a targeted delivery vector according to the second aspect of the present invention or a liposome according to the tenth aspect in the manufacture of a composition for the treatment of a disease.

In a fifteenth aspect, the present invention relates to the use of a liposome coupled to a polymer in the preparation of a non-viral delivery vector comprising siRNA.

In a sixteenth aspect, the present invention relates to the use of a liposome coupled to a polymer and one or more agents in the preparation of a targeted non-viral delivery vector comprising siRNA.

In an seventeenth aspect, the present invention relates to a non-viral delivery vector or a targeted non-viral delivery vector substantially as described herein and with reference to any one of the Examples or Figures.

In an eighteenth aspect, the present invention relates to a method substantially as described herein and with reference to any one of the Examples or Figures.

In a nineteenth aspect, the present invention relates to a use substantially as described herein and with reference to any one of the Examples or Figures.

In a twentieth aspect, the present invention relates to a liposome substantially as described herein and with reference to any one of the Examples or Figures.

### PREFERRED EMBODIMENTS

Preferably, the one or more lipids of the liposome that are coupled, reversibly or irreversibly, to one or more polymers, are exposed at the surface of the liposome.

Preferably, the liposome comprises one or more aminoxy group containing lipids of the formula (I):

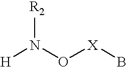

wherein B is a lipid; wherein X is an optional linker group and wherein R₂ is H or a hydrocarbyl group.

Preferably, the aminoxy group containing lipid is cholesteryl-dPEG₄)₂-aminoxy lipid (CPA).

Preferably, the liposome comprises one or more cationic lipids and/or one or more non-cationic co-lipids.

Preferably, the cationic lipid comprises at least one alicyclic group.

Preferably, the at least one alicyclic group is cholesterol.

Preferably, the cationic lipid is N¹-cholesteryloxycarbonyl-3,7-diazanononane-1,9-diamine (CDAN).

Preferably, the non-cationic co-lipid is a phosphatidylethanolamine. More preferably, the non-cationic co-lipid is dioleoyl phosphatidylethanolamine (DOPE).

Preferably, the polymer comprises one or more aldehyde and/ketone groups. More preferably, the polymer is PEG.

Preferably, the liposome is coupled with from about 0.1 to about 5% PEG.

Preferably, the liposome comprises or is coupled, reversibly or irreversibly to, one or more agents.

Preferably, the agent(s) of the targeted non-viral delivery vector are selected from the group consisting of sugar, carbohydrate and a ligand.

Preferably, the sugar is selected from the group consisting of glucose, mannose, lactose, fructose, maltotriose, maltoheptose.

Preferably, the ligand is an antibody.

Preferably, the process according to the sixth aspect of the present invention comprises the additional step of: coupling, reversibly or irreversibly, the liposome formed in step (i) or step (ii) with one or more agent(s).

Preferably, the cryo-protectant is selected from the group consisting of sucrose, trehalose and lactose.

Preferably, the method according to the eighth aspect of the present invention comprises the additional step of: (iv) rehydrating the vector prior to use.

Preferably, the liposome according to the tenth aspect of the present invention is of the formula

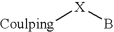

wherein B is a lipid; wherein X is a linker group and Coupling is a coupling moiety, wherein the X backbone comprises at least 30 atoms.

Preferably, the X backbone comprises at least 40 atoms

Preferably, X is or comprises a group of the formula

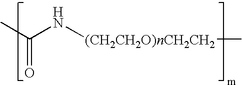

wherein n and m are independently from 0 to 6, preferably from 1 to 6, more preferably 2, 3 or 4, more preferably 2 or 4.

Preferably, X is or comprises a group of the formula

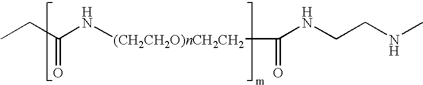

wherein n and m are independently from 0 to 6, preferably from 1 to 6, more preferably 2, 3 or 4, more preferably 2 or 4.

Preferably, X is or comprises a group of the formula

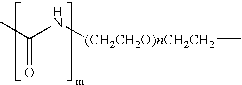

wherein m is from 0 to 6, preferably from 1 to 6, more preferably 1, 2 or 3, more preferably 1 and wherein n is from 0 to 20, preferably from 5 to 15, more preferably 10, 11 or 12, more preferably 11.

Preferably, X is or comprises a group of the formula

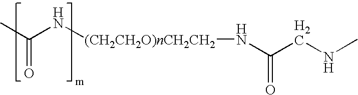

wherein m is from 0 to 6, preferably from 1 to 6, more preferably 1, 2 or 3, more preferably 1 and wherein n is from 0 to 20, preferably from 5 to 15, more preferably 10, 11 or 12, more preferably 11.

Preferably, the disease is liver disease and/or liver damage.

### ADVANTAGES

The present invention has a number of advantages. These advantages will be apparent in the following description.

By way of example, the present invention is advantageous since it provides a method for delivering siRNA using non-viral mediated methods.

By way of further example, the present invention is advantageous since the non-viral delivery vectors described herein are serum resistant and less susceptible to degradation.

By way of further example, the present invention is advantageous since the non-viral delivery vectors are dramatically stabilised against aggregation without impairing the power of the siRNA to downregulate a target gene.

By way of further example, the present invention is advantageous since the non-viral delivery vectors can be coated with a further agent—such as an antibody—to generate a targeted non-viral delivery vector for the delivery of siRNA to a specific site of interest.

### DESCRIPTION OF THE FIGURES

FIG. 1

(a) PEGylated siRNA-lipoplexes generated by post-coupling PEG to an siRNA loaded lipoplex mediate specific down-regulation of a specifically targeted gene in a dose-response dependent manner in vitro, even if the PEG is irreversibly coupled to the surface of the siRNA-lipoplex by means of an oxime bond formed between the aldehydic groups of the PEG and the aminoxy functional group of the aminoxylipid CPA.

(b) This specific down-regulation of a specific gene is dependent on the quantity of PEG coupled to the surface of the siRNA-lipoplex.

FIG. 2

PEGylated siRNA-lipoplexes generated by post-coupling PEG to an siRNA loaded lipoplex exhibit serum stability with increasing amounts of PEG coupled to the surface.

FIG. 3

PEGylated siRNA-lipoplexes generated by post-coupling PEG to an siRNA loaded lipoplex exhibit a pharmacokinetics profile different from the non-pegylated analogue, gradually decreasing the amount detected in the liver with increasing amounts of PEG.

FIG. 4

Surprisingly, the down-regulation of the lacZ gene that was introduced into the liver of female Balb/C mice by hydrodynamic injection of 1 μg pDNA (in 2 ml PBS) reached more than 80% after systemic delivery of 20 μg siRNA-lipoplex (PEG 0.1%) 8 or 24 hours post-hydrodynamic injection.

FIG. 5

Even more unexpectedly, male Balb/C mice that were infected with a given dose of a lacZ-adenovirus and 2 h post-viral infection obtained 20 μg siRNA-lipoplex (PEG 0%/0.1%/5%) via tail vain injection showed maximum downregulation (>70%) of the highest PEGylated siRNA-lipoplex (5% PEG).

FIG. 6

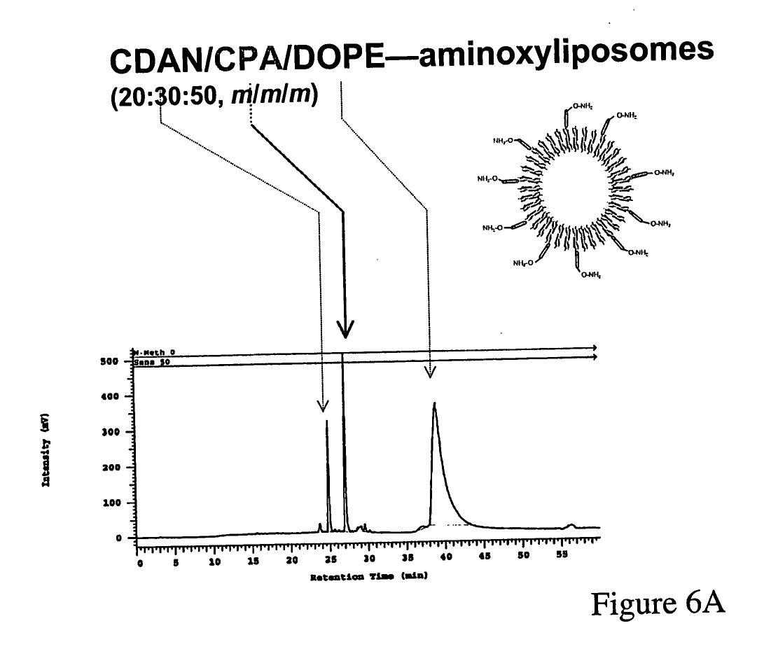

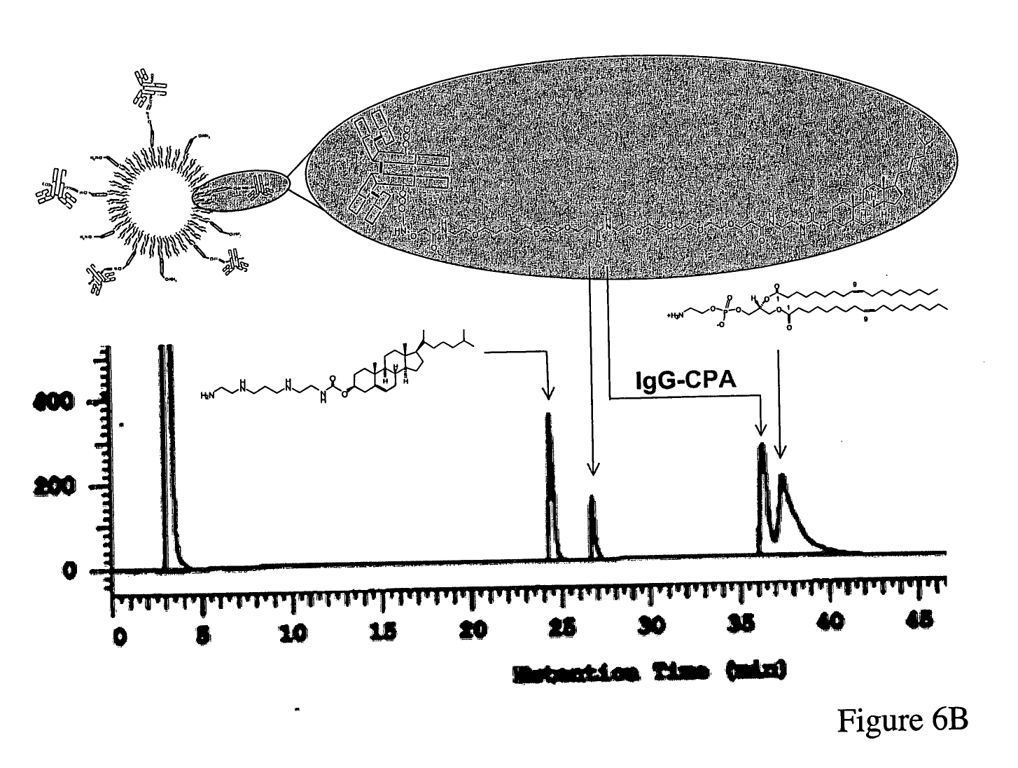

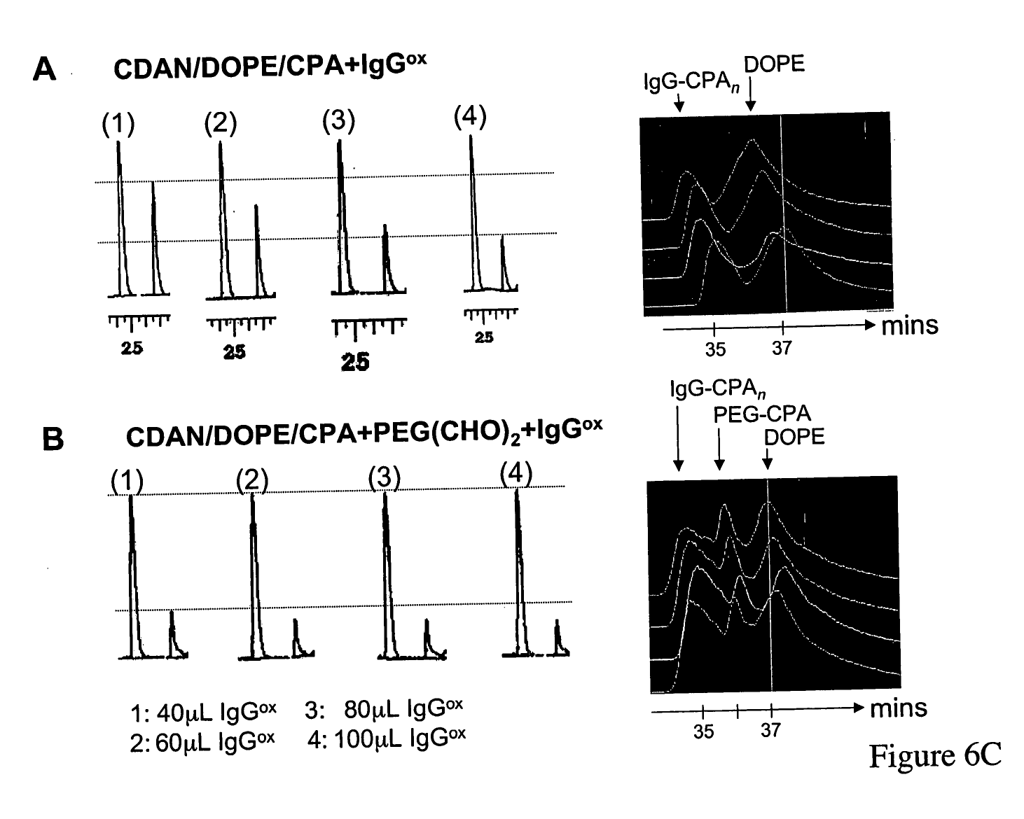

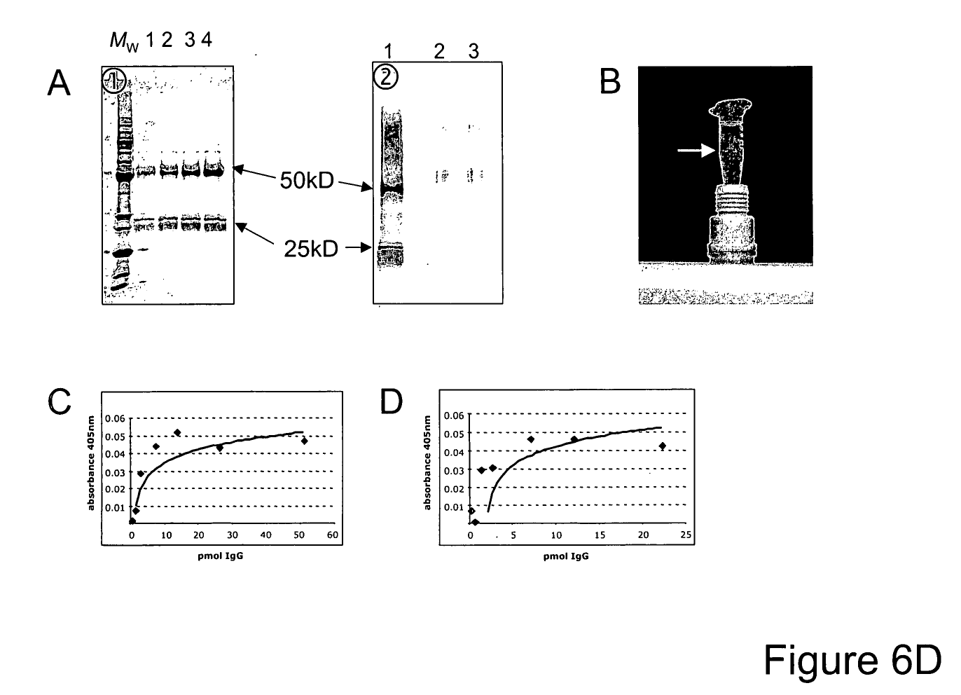

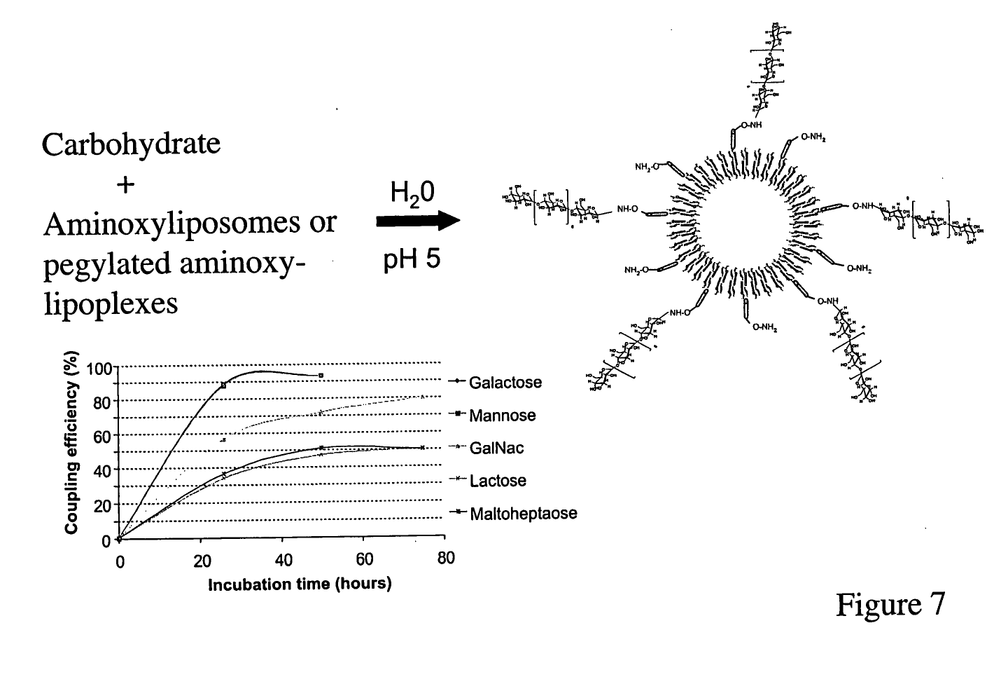

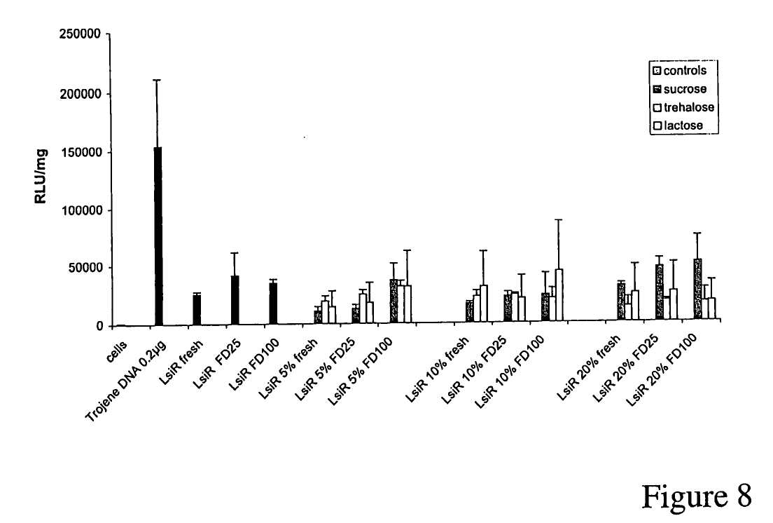

LsiR lipoplexes made from siRNA and CDAN/DOPE/CPA (40/50/10; m/m/m) liposomes pegylated at 0.1-1% total lipid (molar ratio in the lipoplex) can be incubated with an oxidized IgG antibody at acidic pH, resulting in the covalent coupling of the antibody through its partially oxidized carbohydrate units to the CPA lipid as demonstrated by HPLC analyses of aminoxy liposomes before incubation with oxidized IgG (FIG. 6a) and after incubation with oxidized IgG (FIG. 6b). Double incubation of liposomes with (i) PEG²⁰⁰⁰ (CHO)₂ followed by (ii) IgG^{OX} was also performed (FIG. 6c). Referring to FIG. 6d, FIG. 6d (A1) shows the results of an SDS PAGE gel (12.5% Tris/glycine). Mw, molecular weight BenchMark™ Protein Ladder (Invitrogen); Lane 1, native, unoxidized human fibronectin (HFN) IgG; lane 2, oxidation of HFN-IgG for 30 mins/10 mM periodic acid; lane 3, oxidation of HFN-IgG for 60 mins/10 mM periodic acid; lane 4, oxidation of HFN-IgG for 120 mins/10 mM periodic acid; The gel was stained with coomassie blue. FIG. 6d (A2) shows the results of an SDS page gel (12.5 Tris/glycine). Lane 1, native, unoxidized HFN-IgG; lane 2, LsiR lipoplex with covalently coupled HFN-IgG^{OX} after FPLC purification, fraction one; lane 3, LsiR lipoplex with covalently coupled HFN-IgG^{OX} after FPLC purification, fraction two. Note that both FPLC fractions contain antibody whose Fc fragment runs above the 50 kD molecular weight band which is an indication of CPA-lipid coupled to the oxidized carbohydrates of the Fc unit. The two bands in FPLC arise due to different sizes of the lipoplexes with the second fraction exhibiting a considerably higher particle size (10,000 nm) as compared to the first fraction (200 nm), which indicates a state of aggregation. The gel was silver stained using standard procedures to visualize the protein bands. FIG. 6d (B) shows the results of an LsiR lipoplex with covalently coupled HFN-IgG^{OX} after sucrose gradient. The fluorescent band (indicated by an arrow) comes from the fluorescently labelled (Cy3)-siRNA. FIG. 6d(C) shows the results of an ELISA of HFN-IgG^{OX} demonstrating the specific binding of the IgG to human fibronectin (HFN). FIG. 6d (D) shows the results of an ELISA of LsiR lipoplex with covalently coupled HFN-IgG^{OX} after purification in sucrose gradient demonstrating similar binding characteristics as observed with native and oxidized HFN-IgG.

FIG. 7

Natural carbohydrates—such as glucose, mannose, lactose, fructose, maltotriose, and maltoheptaose can be incubated with LsiR lipoplexes made from siRNA and CDAN/DOPE/CPA (40/50/10; m/m/m) liposomes pegylated at 0.1-1% total lipid (molar ratio in the lipoplex) to form a covalent conjugation of the Cl-carbohydrate atom with the aminoxy functional group of the CPA lipid.

FIG. 8

β-galactosidase down-regulation with freeze dried LsiR on HeLa. LsiR fresh, freshly prepared LsiR; LsiR 12 FD 25, LsiR complex freeze dried and rehydrated in 25 μl water, no cryo-protective agent; LsiR 12 FD 100, LsiR complex freeze dried and rehydrated in 100 μl water, no cryo-protective agent. The graph represents comparisons of the three different cryo-protective agents used (sucrose, trehalose or lactose) at 5%/10% or 20% (w/v), rehydrated in either 25 μl (FD25) or 100 μl (FD100) water, respectively.

### DETAILED DESCRIPTION OF THE INVENTION

siRNA

As described above, siRNA is the basis of the so called “RNA induced interference” (RNAi) concept, which is a method of post-transcriptional gene regulation that is conserved throughout many eukaryotic organisms.

RNAi is induced by short (typically less than 30 nucleotides) double stranded RNA molecules which are present in the cell (Fire A et al. (1998), Nature 391: 806-811). These short dsRNA molecules (or siRNA) cause the destruction of messenger RNAs which share sequence homology with the siRNA to within one nucleotide resolution (Elbashir S M et al. (2001), Genes Dev, 15: 188-200). It is believed that the siRNA and the targeted mRNA bind to an RNA-induced silencing complex, which cleaves the targeted mRNA. The siRNA is apparently recycled much like a multiple-turnover enzyme, with 1 siRNA molecule capable of inducing cleavage of approximately 1000 mRNA molecules. siRNA-mediated RNAi degradation of mRNA is therefore highly effective for inhibiting expression of a target gene.

The siRNA described herein may comprise partially purified RNA, substantially pure RNA, synthetic RNA, or recombinantly produced RNA, as well as altered RNA that differs from naturally-occurring RNA by the addition, deletion, substitution and/or modification of one or more nucleotides.

Such alterations can include the addition of non-nucleotide material—such as modified nucleotides—to, for example, the end(s) of the siRNA or to one or more internal nucleotides of the siRNA, including modifications that make the siRNA resistant or even more resistant to nuclease digestion.

A number of different types of modifications are known in the art. These include methylphosphonate and phosphorothioate backbones and/or the addition of acridine or polylysine chains at the 3′ and/or 5′ ends of the molecule. The nucleotide sequences may be modified by any method available in the art. Such modifications may be carried out to enhance the in vivo activity or life span of the siRNA.

One or both strands of the siRNA may comprise a 3′ overhang.

Thus, the siRNA may comprise at least one 3′ overhang of, for example, from 1 to about 6 nucleotides (which includes ribonucleotides or deoxynucleotides) in length. If both strands of the siRNA molecule comprise a 3′ overhang, the length of the overhangs can be the same or different for each strand.

In order to enhance the stability of the siRNA, the 3′ overhangs may be stabilised against degradation. The overhangs may be stabilised by including purine nucleotides—such as adenosine or guanosine nucleotides. Alternatively, substitution of pyrimidine nucleotides by modified analogues may be tolerated and may not affect the efficiency of RNAi degradation.

Typically, the siRNA will be in the form of isolated siRNA comprising short double-stranded RNA from about 17 nucleotides to about 29 nucleotides in length—such as approximately 19-25 contiguous nucleotides in length—that are targeted to a target mRNA. The siRNA comprise a sense RNA strand and a complementary antisense RNA strand annealed together by standard Watson-Crick base-pairing interactions. The sense strand comprises a nucleic acid sequence which is identical to a target sequence contained within the target mRNA.

As used herein, the term “isolated siRNA” means that the siRNA is altered or removed from the natural state through human intervention. An isolated siRNA can exist in substantially purified form, or can exist in a non-native environment such as, for example, a cell into which the siRNA has been delivered.

The sense and antisense strands of the siRNA can comprise two complementary, single-stranded RNA molecules or can comprise a single molecule in which two complementary portions are base-paired and are covalently linked by a single-stranded hairpin.

It is understood that human mRNA may contain target sequences in common with their respective alternative splice forms, cognates or mutants. A single siRNA comprising such a common targeting sequence can therefore induce RNAi-mediated degradation of different RNA types which contain a common targeting sequence.

A target sequence on the target mRNA may be selected from a given sequence—such as a cDNA sequence—corresponding to the target mRNA, using various methods in the art. For example, the rational design of siRNAs is described in Nat Biotechnol. (2004) 22(3):326-30. siRNAs can be designed based on the following guidelines. Firstly, a sequence of around 21 nucleotides in the target mRNA is identified that begins with an AA dinucleotide. Each AA is recorded and the 3′ adjacent nucleotides are identified as potential siRNA target sites. This is based on the observation by Elbashir et al. (EMBO J (2001) 20: 6877-6888, Nature (2001) 411: 494-498. 2 and Genes & Dev. (2001) 15: 188-200) that siRNAs with 3′ overhanging UU dinucleotides are the most effective. However, siRNAs with other 3′ terminal dinucleotide overhangs have been shown to effectively induce RNAi. Preferably, target sites from among the sequences identified above are then further selected using one or more the following criteria: (i) selecting siRNAs with 30-50% GC content; (ii) avoid stretches of >4 T's or A's in the target sequence; (iii) select siRNA target sites at different positions along the length of the gene sequence; and (iv) eliminate any target sequences with more than 16-17 contiguous base pairs of homology to other coding sequences.

siRNA sequences may even be derived from an algorithm that verifies off-target down-regulation as described in Kumiko Ui-Tei et al, Nucl. Acids Res. 2004, Vol 32, No. 3, p. 936-48).

If the selected siRNA sequences does not function for silencing, the following steps may be used. A search may be conducted for sequencing errors in the gene and possible polymorphisms. Studies on the specificity of target recognition by siRNA indicate that a single point mutation located in the paired region of an siRNA duplex is sufficient to abolish target mRNA degradation. A second and/or third target may also be selected and the corresponding siRNA prepared and tested.

Although siRNA silencing is highly effective by selecting a single target in the mRNA, it may be desirable to design and employ two independent siRNA duplexes to control the specificity of the silencing effect.

siRNA may be obtained using a number of techniques known to those of skill in the art. For example, the siRNA may be chemically synthesized using appropriately protected ribonucleoside phosphoramidites and a conventional DNA/RNA synthesiser. The siRNA may be synthesized as two separate, complementary RNA molecules, or as a single RNA molecule with two complementary regions.

Further methods for the design of siRNA may be found at the websites of, for example, QIAGEN, Ambion and Ocimum Biosolutions.

SiRNA may also be purchased from several companies—such as Dharmacon (USA) and Qiagen GmbH (Hilden, Germany).

The siRNA may even be labelled. By way of example, the siRNA may be labelled with a 3′-FITC label anti-GFP.

siRNA may be recombinantly produced using methods known in the art. For example, siRNA may be expressed from recombinant circular or linear DNA plasmids using any suitable promoter. The recombinant plasmids of the invention can also comprise inducible or regulatable promoters for expression of the siRNA in a particular tissue or in a particular intracellular environment.

The siRNA expressed from recombinant plasmids can either be isolated from cultured cell expression systems by standard techniques. siRNA may be expressed from a recombinant plasmid either as two separate, complementary RNA molecules, or as a single RNA molecule with two complementary regions.

Selection of plasmids suitable for expressing siRNA of the invention, methods for inserting nucleic acid sequences for expressing the siRNA into the plasmid, and methods of obtaining the siRNA are described in, for example, Science (2002) 296: 550-553; Nat. Biotechnol. (2002) 20: 497-500; Genes Dev. (2002), 16: 948-958; Nat. Biotechnol. (2002) 20: 500-505; and Nat. Biotechnol. (2002) 20: 505-508.

siRNA sequences may include those that are of therapeutic and/or diagnostic application—such as sequences encoding cytokines, chemokines, hormones, antibodies, engineered immunoglobulin-like molecules, a single chain antibody, fusion proteins, enzymes, immune co-stimulatory molecules, immunomodulatory molecules, a transdominant negative mutant of a target protein, a toxin, a conditional toxin, an antigen, a tumour suppressor protein, a growth factor, a membrane protein, a vasoactive proteins and peptides, an anti-viral protein and/or a ribozyme.

The target mRNA may be or may be derived from the anti-apoptotic protein livin-2 (U73857), which is used for stimulating caspase-3, resulting in the onset of apoptosis in the cell line transfected with siRNA. One example of such a siRNA sequence which targets this mRNA is 5′-GGG CGU GGU GGG UUC UUG AGC-3′.

The target mRNA may be or may be derived from HBV, HCV and/or P-pg.

The siRNA may be targeted to a target mRNA that is or is derived from HBV, HCV and/or P-glycoprotein.

In particular, the sequences of Hepatitis B virus include Hepatitis B virus isolate 2-AII-BR large S protein (S) gene (Accession number AY344099.1); Hepatitis B virus isolate 6-AIII-BR large S protein (S) gene (Accession number AY344104.1); Hepatitis B virus isolate j13 small surface protein (S) gene (Accession number AY639927.1); Hepatitis B virus isolate j7 small surface protein (S) gene (Accession number AY639924.1); Hepatitis B virus isolate 17993 (Accession number AY217367.1); and/or Hepatitis B virus isolate Q7-1 (Accession number AY217365.1).

Preferably, the HBV siRNA sequences are directed against the conserved sequence of the HBV core gene. More preferably, the HBV siRNA sequences are selected from the group consisting of: IA1: 5′-GTCGTCCTTTCTCGGAAAT; IA2: 5′-ACTCATCGGGACTGATAAT; and IA3: 5′-GCGGGACGTCCTTTGTTTA. The sequences were obtained using the GPboost algorithm directed against the conserved sequence of the HBV core gene. All of these 19 nt RNA sequences were chemically synthesized by Dharmacon (Colorado, USA) with two DNA base pairs dTdT overhangs at both 3′ strands. All sequences were PAGE purified. Sequence IA3 revealed a potent pattern of downregulation of the HBV surface antigen in vitro and in vivo (results not shown).

Hepatitis C virus is one of the main causes of liver-related morbidity and mortality. The virus establishes a persistent infection in the liver, leading to the development of, for example, chronic hepatitis, liver cirrhosis and hepatocellular carcinomas. A satisfactory treatment has not yet been developed, and the current treatment, interferon in combination with ribavirin fails in nearly 50% of patients. The HCV virus is a positive stranded RNA virus containing a single, long open reading frame that encodes structural and non-structural proteins. Translation of the viral genome is mediated by an internal ribosomal entry site (IRES) which is located in the untranslated region at the 5′ terminus (5′-UTR; Accession Number D31603), which is also conserved in 99.6% of all virus strains. Therefore, it constitutes an ideal target for an siRNA drug. Similarly, the 3′-UTR (accession No D63922) is highly conserved and has been demonstrated to exhibit an important role for the virus replication in vivo.

The sequences of Hepatitis C virus include human hepatitis virus C capsid and envelope:x proteins (Accession number M55970.1); the 5′-UTR region (341 nt) that is conserved throughout all HCV isolates (Accession number M55970.1); and/or non structural proteins—such as NS3, NS4, NS5A and NS5B

The sequences of Hepatitis C virus include modified forms of hepatitis C virus NS3 protease (Accession number BD270935.1).

Preferably, the one or more siRNA sequences are directed towards the untranslated region at the 5′ terminus (5′-UTR; Accession Number D31603) or the 3′-UTR (Accession No D63922) of HCV. More preferably, the HCV sequences are selected from the group consisting of:

HCVIA146:
5′-GTCACGGCTAGCTGTGAAAdTdT;
HCVIA185:
5′-TGCAGAGAGTGCTGATACTdTdT;
HCVIA205:
5′TGGCCTCTCTGCAGATCATdTdT;
HCVIA56-5′-UTR:
5′TACTGTCTTCACGCAGAAAdTdT;
HCVIA210-5′-UTR
5′CGCTCAATGCCTGGAGATTdTdT;
HCVIA211-5′-UTR
5′GCTCAATGCCTGGAGATTTdTdT;
and
HCVIA258-5′-UTR:
5′-GTAGTGTTGGGTCGCGAAAdTdT.

All of the these 19 nt RNA sequences were chemically synthesised by Dharmacon (Colorado, USA) with two DNA base pairs dTdT overhangs at both 3′ strands. All sequences were PAGE purified. The efficacies of the individual sequences were compared to siRNA331 (5′-GGUCUCGUAGACCGUGCAC) described by Yokota et al, EMBO Reports 4, 6, 2003, 602ff.

The sequences of P-glycoprotein (MDR1 gene product) includes the Homo sapiens P-glycoprotein (ABCB1) (Accession number AF399931.1).

Also included within the scope of the present invention are variants, homologues, fragments and derivatives of the nucleotide sequences described herein.

In a preferred embodiment, the siRNA is in the form of isolated siRNA comprising short double-stranded RNA from, for example, about 17 nucleotides to about 29 nucleotides in length—such as approximately 19-25 contiguous nucleotides in length—that are targeted to a target mRNA that is or is derived from HBV, HCV and P-pg protein.

Liposome

Liposomes are typically completely closed structures comprising lipid bilayer membranes containing an encapsulated aqueous volume. Liposomes may contain many concentric lipid bilayers separated by an aqueous phase (multilamellar vesicles or MLVs), or alternatively, they may comprise a single membrane bilayer (unilamellar vesicles). The lipid bilayer is composed of two lipid monolayers having a hydrophobic “tail” region and a hydrophilic “head” region. In the membrane bilayer, the hydrophobic (nonpolar) “tails” of the lipid monolayers orient toward the centre of the bilayer, whereas the hydrophilic (polar) “heads” orient toward the aqueous phase.

The lipid components that may be used in the liposomes described herein are generally described in the literature. Generally, these are phospholipids—such as phosphatidylcholine, phosphatidylethanolamine, phosphatidylglycerol, phosphatidylserine, phosphatidic acid, phosphatidylinositol and/or sphingolipids. Additional components, fore example, sterols—such as cholesterol—or other components—such as fatty acids (e.g., stearic acid, palmitic acid), dicetyl phosphate or cholesterol hemisuccinate, may be used. Moreover, the liposome membrane can also contain preservatives. The liposome membrane may also contain components, which modify their dispersion behaviour. They include, for example, PEGylated derivatives of phosphatidylethanolamine, lipids—such as GM 1—or conjugates of sugars and hydrophobic components—such as palmitic or stearic acid esters of dextran.

The basic structure of liposomes may be made by a variety of techniques known in the art.

For example, liposomes have typically been prepared using the process of Bangham et al., (1965 J. Mol. Biol., 13: 238-252), whereby lipids suspended in organic solvent are evaporated under reduced pressure to a dry film in a reaction vessel. An appropriate amount of aqueous phase is then added to the vessel and the mixture agitated. The mixture is then allowed to stand, essentially undisturbed for a time sufficient for the multilamellar vesicles to form.

Liposomes may be reproducibly prepared using a number of currently available techniques. The types of liposomes which may be produced using a number of these techniques include small unilamellar vesicles (SUVs) [See Papahadjapoulous and Miller, Biochem. Biophys. Acta., 135, p. 624-638 (1967)], reverse-phase evaporation vesicles (REV) [See U.S. Pat. No. 4,235,871 issued Nov. 25, 1980], stable plurilamellar vesicles (SPLV) [See U.S. Pat. No. 4,522,803, issued Jun. 11, 1985], and large unilamellar vesicles produced by an extrusion technique as described in copending U.S. patent application Ser. No. 622,690, filed Jun. 20, 1984, Cullis et. al., entitled “Extrusion Technique for Producing Unilamellar Vesicles”.

In a preferred embodiment, the liposomes are prepared using the following method. The lipids are prepared by pipetting the appropriate amount of stock solutions of, for example, CDAN, DOPE and aminoxylipid (CPA), respectively, into a round bottomed flask pre-treated with nitric acid and dimethylsilyldichlorid, evaporating the solvent, and hydrating the dry lipid film with water under heavy vortexing, to generate multilamellar liposomes.

Unilamellar liposomes may be produced by sonicating the multilamellar liposomes for 30 mins. Preferably, this is continued until a size of smaller than about 30 nm is reached.

To add the siRNA to the liposomes, a solution of siRNA in water is added drop-wise to these liposomes under heavy vortexing. Preferably, this is continued until a final siRNA concentration of about 0.1 mg/mL is reached.

The resulting siRNA lipoplex typically measures about 30-50 nm diameter as determined by PCS.

The vectors described herein may be lyophilised. Preferably, the vectors are freeze dried. Thus, in a further aspect there is provided a method comprising the steps of: (i) providing a vector as described herein; (ii) optionally, contacting the vector with a cryo-protectant; and (iii) freeze-drying the vector.

Advantageously, the freeze-dried vectors can be stored over a prolonged period of time. Following storage, the vectors can be rehydrated in water prior to use.

Advantageously, freeze drying in the presence of a cryogenic agent—such as sucrose, trehalose and lactose—does not impair the vectors in terms of size (PCS) and activity. Surprisingly, it has been found that the vectors that are freeze-dried in the presence of trehalose are even more potent than freshly prepared vectors.

The polymer is added using a solution of, for example, polyethyleneglycol-β/γ-bisaldehyde (Mw 2000, or 3400, respectively; NEKTAR, USA) at, for example, 1 mg/mL. The appropriate amount of the polymer is then added to the siRNA lipoplex under vortexing. Typically, this will result in covalently surface-pegylated siRNA-lipoplexes with differential amounts of polymer (for example, 0.1-5%, m/m total lipid). Half of the volume may be evaporated under reduced pressure and compensated by addition of PBS prior to use, e.g. injection into animals.

It may also be desirable to include other ingredients in the liposome—such as diagnostic markers including radiolabels, dyes, chemiluminescent and fluorescent markers; contrasting media; imaging aids; agents and so forth.

The liposome preferably comprises one or more aminoxy group containing lipids of the formula (I):

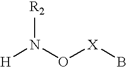

wherein B is a lipid; wherein X is an optional linker group and wherein R₂ is H or a hydrocarbyl group. Preferred lipids are those in International (PCT) Patent Application No: PCT/GB01/05385.

Here, the term “hydrocarbyl group” in the context of formula (I) means a group comprising at least C and H and may optionally comprise one or more other suitable substituents. Examples of such substituents may include halo, alkoxy, nitro, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. A non-limiting example of a hydrocarbyl group is an acyl group.

A typical hydrocarbyl group is a hydrocarbon group. Here the term “hydrocarbon” means any one of an alkyl group, an alkenyl group, an alkynyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

The hydrocarbyl/hydrocarbon/alkyl may be straight chain or branched and/or may be saturated or unsaturated.

In one preferred aspect the hydrocarbyl/hydrocarbon/alkyl may be selected from straight or branched hydrocarbon groups containing at least one hetero atom in the group.

In one preferred aspect the hydrocarbyl/hydrocarbon/alkyl may be a hydrocarbyl group comprising at least two carbons or wherein the total number of carbons and hetero atoms is at least two.

In one preferred aspect the hydrocarbyl/hydrocarbon/alkyl may be selected from hydrocarbyl groups containing at least one hetero atom in the group. Preferably the hetero atom is selected from sulphur, nitrogen and oxygen.

In one preferred aspect the hydrocarbyl/hydrocarbon/alkyl may be selected from straight or branched hydrocarbon groups containing at least one hetero atom in the group. Preferably the hetero atom is selected from sulphur, nitrogen and oxygen.

In one preferred aspect the hydrocarbyl/hydrocarbon/alkyl may be selected from straight or branched alkyl groups, preferably C₁₋₁₀ alkyl, more preferably C₁₋₅ alkyl, containing at least one hetero atom in the group. Preferably the hetero atom is selected from sulphur, nitrogen and oxygen.

In one preferred aspect the hydrocarbyl/hydrocarbon/alkyl may be selected from straight chain alkyl groups, preferably C₁₋₁₀ alkyl, more preferably C₁₋₅ alkyl, containing at least one hetero atom in the group. Preferably the hetero atom is selected from sulphur, nitrogen and oxygen.

The hydrocarbyl/hydrocarbon/alkyl may be selected from
C₁-C₁₀ hydrocarbyl,
C₁-C₅ hydrocarbyl
C₁-C₃ hydrocarbyl.
hydrocarbon groups
C₁-C₁₀ hydrocarbon
C₁-C₅ hydrocarbon
C₁-C₃ hydrocarbon.
alkyl groups
C₁-C₁₀ alkyl
C₁-C₅ alkyl
C₁-C₃ alkyl

The hydrocarbyl/hydrocarbon/alkyl may be straight chain or branched and/or may be saturated or unsaturated.

The hydrocarbyl/hydrocarbon/alkyl may be straight or branched hydrocarbon groups containing at least one hetero atom in the group.

Preferably R₂ is H or a hydrocarbyl group.

In a preferred aspect the R₂ hydrocarbyl group contains optional heteroatoms selected from O, N and halogens.

In a preferred aspect R₂ is H.

Preferably, the lipid is, or is derived from, or comprises a cholesterol group

The cholesterol group may be or may be derived from cholesterol or a derivative thereof.

Examples of cholesterol derivatives include substituted derivatives wherein one or more of the cyclic CH₂ or CH groups and/or one or more of the straight-chain CH₂ or CH groups is/are appropriately substituted. Alternatively, or in addition, one or more of the cyclic groups and/or one or more of the straight-chain groups may be unsaturated.

In a preferred embodiment the cholesterol group is cholesterol.

In a preferred aspect optional linker X is present.

In a preferred aspect X is a hydrocarbyl group.

In a preferred aspect the linker X comprises or is linked to the lipid via a polyamine group.

It is believed that the polyamine group is advantageous because it increases the DNA binding ability and efficiency of gene transfer of the resultant liposome.

In one embodiment, preferably the polyamine group is a unnaturally occurring polyamine. Preferably two or more of the amine groups of the polyamine group of the present invention are separated by one or more groups which are not found in nature that separate amine groups of naturally occurring polyamine compounds (i.e. preferably the polyamine group of the present invention has un-natural spacing).

Preferably the polyamine group contains at least two amines of the polyamine group that are separated (spaced from each other) from each other by an ethylene (—CH₂CH₂—) group.

Preferably each of the amines of the polyamine group are separated (spaced from each other) by an ethylene (—CH₂CH₂—) group.

In one preferred aspect X is or comprises a group of the formula

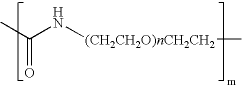

wherein n and m are independently from 0 to 6, preferably from 1 to 6, more preferably 2, 3 or 4, more preferably 2 or 4. In a highly preferred aspect m is 2 and n is 4.

In one preferred aspect X is or comprises a group of the formula

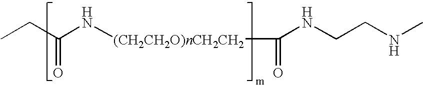

wherein n and m are independently from 0 to 6, preferably from 1 to 6, more preferably 2, 3 or 4, more preferably 2 or 4. In a highly preferred aspect m is 2 and n is 4.

In one preferred aspect the liposome is of the formula

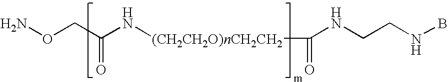

wherein B is a lipid and wherein n and m are independently from 0 to 6, preferably from 1 to 6, more preferably 2, 3 or 4, more preferably 2 or 4. In a highly preferred aspect m is 2 and n is 4.

In one preferred aspect X is or comprises a group of the formula

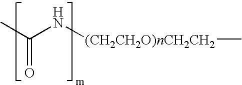

wherein m is from 0 to 6, preferably from 1 to 6, more preferably 1, 2 or 3, more preferably 1 and wherein n is from 0 to 20, preferably from 5 to 15, more preferably 10, 11 or 12, more preferably 11.

In one preferred aspect X is or comprises a group of the formula

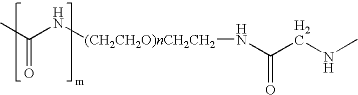

wherein m is from 0 to 6, preferably from 1 to 6, more preferably 1, 2 or 3, more preferably 1 and wherein n is from 0 to 20, preferably from 5 to 15, more preferably 10, 11 or 12, more preferably 11.

In one preferred aspect the liposome is of the formula

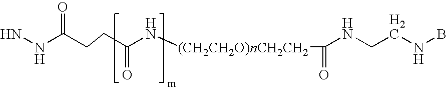

wherein B is a lipid, wherein m is from 0 to 6, preferably from 1 to 6, more preferably 1, 2 or 3, more preferably 1 and wherein n is from 0 to 20, preferably from 5 to 15, more preferably 10, 11 or 12, more preferably 11.

Typical examples of suitable polyamines include spermidine, spermine, caldopentamine, norspermidine and norspernine. An alternative preferred polyamine is caldopentamine.

In a highly preferred embodiment, the aminoxy group containing lipid is CPA.

Preferably, the liposome comprises one or more cationic lipids.

A variety of cationic lipids are known in the art. Example structures of such cationic lipids are provided in Table 1 of WO95/02698.

Generally, any cationic lipid, either monovalent or polyvalent, may be used.

Polyvalent cationic lipids are generally preferred.

Cationic lipids include saturated and unsaturated alkyl and alicyclic ethers and esters of amines, amides or derivatives thereof. Straight-chain and branched alkyl and alkene groups of cationic lipids can contain from 1 to about 25 carbon atoms. Preferred straight-chain or branched alkyl or alkene groups have six or more carbon atoms. Alicyclic groups can contain from about 6 to 30 carbon atoms. Preferred alicyclic groups include cholesterol and other steroid groups. Cationic lipids can be prepared with a variety of counterions (anions) including among others: chloride, bromide, iodide, fluoride, acetate, trifluoroacetate, sulfate, nitrite, and nitrate.

A well-known cationic lipid is N-[1-(2,3-dioleyloxy)propyl]-N,N,N-trimethylammonium chloride (DOTMA).

DOTMA and the analogous diester DOTAP (1,2-bis(oleoyloxy)-3 (trimethylammonium) propane), are commercially available.

Additional cationic lipids structurally related to DOTMA are described in U.S. Pat. No. 4,897,355.

Another useful group of cationic lipids related to DOTMA and DOTAP are commonly called DORI-ethers or DORI-esters. DORI lipids differ from DOTMA and DOTAP in that one of the methyl groups of the trimethylammonium group is replaced with a hydroxyethyl group. The oleoyl groups of DORI lipids can be replaced with other alkyl or alkene groups, such as palmitoyl or stearoyl groups. The hydroxyl group of the DORI-type lipids can be used as a site for further functionalization, for example for esterification to amines, like carboxyspermine.

Additional cationic lipids which can be employed in the delivery vectors or complexes of this invention include those described in WO91/15501 as useful for the transfection of cells.

Cationic sterol derivatives, like 3β[N-(N′,N′-dimethylaminoethane)carbamoyl] cholesterol (DC-Chol) in which cholesterol is linked to a trialkyammonium group, can also be employed in the present invention. DC-Chol is reported to provide more efficient transfection and lower toxicity than DOTMA-containing liposomes for some cell lines. DC-Chol polyamine variants such as those described in WO97/45442 may also be used.

Polycationic lipids containing carboxyspermine are also useful in the delivery vectors or complexes of this invention. EP-A-304111 describes carboxyspermine containing cationic lipids including 5-carboxyspermylglycine dioctadecyl-amide (DOGS) and dipalmitoylphosphatidylethanolamine 5-carboxyspermylamide (DPPES). Additional cationic lipids can be obtained by replacing the octadecyl and palmitoyl groups of DOGS and DPPES, respectively, with other alkyl or alkene groups.

In a preferred embodiment the cationic lipid comprises at least one saturated or unsaturated alicyclic ether or ester of amine, amide or a derivatives thereof. Alicyclic groups can contain from about 6 to 30 carbon atoms. A highly preferred alicyclic group is cholesterol.

Thus, in a highly preferred embodiment, the polycationic lipid is a cholesterol-based lipid.

Preferably, the cholesterol-based lipid is N-cholesteryloxycarbonyl-3,7-diazanononane-1,9-diamine (CDAN)

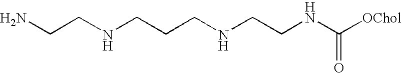

where Chol denotes a group of the formula

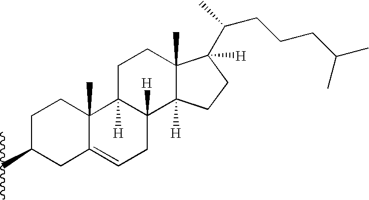

Preferably, the liposomes described herein include non-cationic co-lipids, preferably neutral lipids, to form liposomes or lipid aggregates.

Neutral lipids useful in this invention include, among many others: lecithins; phosphatidylethanolamines, such as DOPE (dioleoyl phosphatidylethanolamine), POPE (palmitoyloleoylphosphatidylethanolamine) and DSPE (distearoylphosphatidylethanol amine); phosphatidylcholine; phosphatidylcholines, such as DOPC (dioleoyl phosphatidylcholine), DPPC (dipalmitoylphosphatidylcholine) POPC (palmitoyloleoyl phosphatidylcholine) and DSPC (distearoylphosphatidylcholine); phosphatidylglycerol; phospha-tidylglycerols, such as DOPG (dioleoylphosphatidylglycerol), DPPG (dipalmitoylphosphatidylglycerol), and DSPG (distearoylphosphatidylglycerol); phosphatidylserines, such as dioleoyl- or dipalmitoylphospatidylserine; diphospha tidylglycerols; fatty acid esters; glycerol esters; sphingolipids; cardolipin; cerebrosides; and ceramides; and mixtures thereof. Neutral lipids also include cholesterol and other 3DOH-sterols.

Preferably, the non-cationic co-lipid is phosphatidylethanolamines. More preferably, the non-cationic co-lipid is DOPE (dioleoyl phosphatidylethanolamine).

In one preferred aspect the lipid is a lipid suitable for use in imaging applications. The imaging lipid may a lipid selected from fluorescent lipids, magnetic resonance imaging lipids, nuclear magnetic resonance imaging lipids, electron microscopy and image processing lipids, electron spin resonance lipids and radioimaging lipids. Suitable and preferred lipids in each of these classes are given below:

Fluorescent Lipids

e.g. 1,2-Dioleoyl-sn-Glycero-3-Phosphoethanolamine-N-(5-dimethylamino-1-naphthalenesulfonyl, 1,2-Dioleoyl-sn-Glycero-3-Phosphoethanolamine-N-(1-pyrenesulfonyl), 1,2-Dioleoyl-sn-Glycero-3-Phosphoethanolamine-N-(Carboxyfluorescein), 1-Oleoyl-2-[6-[(7-nitro-2-1,3-benzoxadiazol-4-yl)amino]hexanoyl]-sn-Glycero-3-Phospho-L-Serine, 25-{N-[(7-nitrobenz-2-oxa-1,3-diazol-4-yl)-methyl]amino}-27-norcholesterol, -Oleoyl-2-[6-[(7-nitro-2-1,3-benzoxadiazol-4-yl)amino]hexanoyl]-sn-Glycero-3-Phosphoethanolamine, 1,2-Dioleoyl-sn-Glycero-3-Phosphoethanolamine-N-(Lissamine Rhodamine B Sulfonyl).

Lipids for Magnetic Resonance Imaging and Nuclear Magnetic Resonance Imaging

Gd-DTPA-bis(stearylamide) (Gd-BSA); Gd-DTPA-bis(myrisitylamide) (GdDTPA-BMA), 1,2-Dimyristoyl-sn-Glycero-3-PhosphoEthanolamineDiethylene-TriaminePentaAcetate: Gd³⁺ (DMPEDTPA:Gd³⁺); D₃₅-1,2-Dihexanoyl-sn-Glycero-3-Phosphocholine

Electron Microscopy and Image Processing

1,2-Dioleoyl-sn-Glycero-3-{[N(5-Amino-1-Carboxypentyl)iminodiAcetic Acid]Succinyl}-(Nickel Salt),

Electron Spin Resonance

1,2-Diacyl-sn-Glycero-3-Phosphotempocholine, 1-Palmitoyl-2-Stearoyl(n-DOXYL)-sn-Glycero-3-Phosphocholine.

Radioimaging

(99m)Tc-DTPA-bis(stearylamide); (99m)Tc-DTPA-bis(myrisitylamide).

Moreover, one or more amphiphilic compounds can optionally be incorporated in order to modify its surface property. Amphiphilic compounds useful in this invention include, among many others; neoglycolipids such as GLU4 and GLU7, polyethyleneglycol lipids such as N-(O-methoxy(polyoxyethylene)oxycarbonyl)-phosphatidylethanolamine, N-monomethoxy (polyoxyethylene) succinylphosphatidylethanol-amine and polyoxyethylene cholesteryl ether; nonionic detergents such as alkyl glycosides, alkyl methyl glucamides, sucrose esters, alkyl polyglycerol ethers, alkyl polyoxyethylene ethers and alkyl sorbitan oxyethylene ethers and steroidal oxyethylene ethers; block copolymers such as polyoxyethylene polyoxypropylene block copolymers.

Accordingly, in a preferred embodiment of the present invention, the liposome comprises one or more aminoxy-group containing lipids, one or more cationic lipids (more preferably, one or more polycationic lipids), and one more non-cationic co-lipids (more preferably neutral lipids).

More preferably, the liposome comprises one or more aminoxy group containing lipids of the formula (I) (preferably CPA) and/or a mixture thereof; one or more polycationic lipids selected from the group consisting of DOTMA, DOTAP, DORI-ethers or DORI-esters, (DC-Chol), DOGS, DPPES, and/or CDAN, and/or mixtures thereof; and one or more neutral lipids selected from the group consisting of DOPE, POPE, DSPE, DOPC, DPPC, DSPC, DOPG, DPPG DSPG, phosphatidylserines, diphospha tidylglycerols, fatty acid esters, glycerol ester, sphingolipids, cardolipin, cerebrosides; and/or ceramides; and/or mixtures thereof.

More preferably, the liposome comprises one or more of the lipids selected from the group consisting of CPA, CDAN and DOPE.

Most preferably, the liposome comprises CPA, CDAN and DOPE.

We have surprising found that particular liposomes for use in accordance with the present invention have particular advantages. These advantages are applicable no only to the present delivery vector comprising siRNA but to a wide range of systems. We have identified that the present liposomes comprising a lipid linked to a coupling moiety via a linker which has a minimum length allows for the preparation of targeted delivery vectors. In particular such a liposome may be used to prepare a vector in which some liposomes are coupled to one or more polymers and the coupling moieties of liposomes having the linker of minimum length (“a long linker”) project beyond the shell created by the polymer. The projecting coupling moieties of the liposomes comprising the long linker may then be used to couple to additional groups, for example the projecting coupling moieties may be used to couple to targeting moieties such as antibodies.

Thus in a further aspect the present invention provides a liposome comprising a lipid and a coupling moiety wherein the distance between the lipid and the coupling moiety is at least 1.5 nm. Preferably the distance between the lipid and the coupling moiety is at least 2 nm, such as least 3 nm or at least 5 nm.

In a further aspect the present invention provides a liposome of the formula

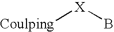

wherein B is a lipid; wherein X is a linker group and Coupling is a coupling moiety, wherein the X backbone comprises at least 30 atoms.

By the term “X backbone” it is meant the shortest chain of directly bonded atoms within the X moiety between the attachment of X to Coupling and the attachment of X to B.

Preferably the X backbone comprises at least 40 atoms, such as at least 50 atoms, such as least 60 atoms.

Preferably the X backbone comprises at least 40 carbon atoms, such as at least 50 carbon atoms, such as least carbon 60 atoms.

By way of example, cholesterylaminoxy lipid 4 as described herein is unsuccessful in conjugating oxidized IgG antibody since the spacer between the cholesteryl moiety and the aminoxy functional group is not long enough. However, it successfully couples polyethyleneglycol-bisaldehyde (Mw 2000 and 3400).

Lipid B may be any lipid described herein. Particularly preferred is a lipid selected from phospholipids such as distearoylphosphatidylethanolamine (DSPE), steroid-based lipids such as cholesterol, amine-based lipids such as dicyclohexylamine.

Linker X may be any linker described herein. It will be understood that by linker it is typically meant a non-reactive linker. Preferably the linker is hydrophilic. The linker may be
a hydrocarbyl group such as an octadecyl group.
peptide-derived such as a polyglycine.
a polymers such as poly(ethylene carbonate), polyethylene glycol (PEG), N-(2-hydroxypropyl)methacrylamide (HMPA), poly (D,L-lactide-co-glycolide) (PLGA), poly (D,L-lactide) (PLA), and poly (glycolide) (PGA).
a carbohydrates such as a dextran, polymannose, hyaluronic acid, oligosaccharides, dextran, pullulan.

In some aspects the linker may also be chemically reactive itself, for example the linker may be peptide derived such as a poly(lysine) (PL) or poly(glutamic acid).

In one preferred aspect X is or comprises a group of the formula

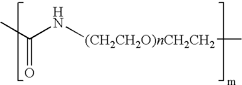

wherein n and m are independently from 0 to 6, preferably from 1 to 6, more preferably 2, 3 or 4, more preferably 2 or 4. In a highly preferred aspect m is 2 and n is 4.

In one preferred aspect X is or comprises a group of the formula

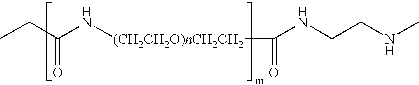

wherein n and m are independently from 0 to 6, preferably from 1 to 6, more preferably 2, 3 or 4, more preferably 2 or 4. In a highly preferred aspect m is 2 and n is 4.

In one preferred aspect X is or comprises a group of the formula

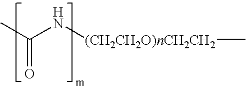

wherein m is from 0 to 6, preferably from 1 to 6, more preferably 1, 2 or 3, more preferably 1 and wherein n is from 0 to 20, preferably from 5 to 15, more preferably 10, 11 or 12, more preferably 11.

In one preferred aspect X is or comprises a group of the formula

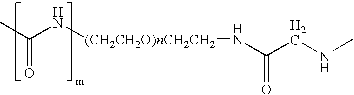

wherein m is from 0 to 6, preferably from 1 to 6, more preferably 1, 2 or 3, more preferably 1 and wherein n is from 0 to 20, preferably from 5 to 15, more preferably 10, 11 or 12, more preferably 11.

Coupling may be any coupling moiety described herein. Particularly preferred is: Nucleophilic groups, in particular amine, thiol, alcohol, aminoxy, hydrazine, hydrazide, azides

Electrophilic groups, in particular isothiocyanate, aldehydes, ketones, isocyanates, maleimide, Michael receptors in general, halides, tosylates, chemical leaving groups in general, actives esters.

Photoligative groups, in particular azides.

Polymer

As used herein, the term “polymer” refers to any polymer that comprises one or more functional groups that interact, bind or are coupled with one or more lipids contained in a liposome.

The polymer may be a naturally occurring polymer or a derivative thereof.

The polymer may be a chemically modified polymer in which the polymer has been modified to include one or more functional groups.

In a preferred embodiment, one or more lipids of the liposome are coupled to one or more polymers. Advantageously, the lipid(s) that are coupled to the polymer(s) are exposed at the surface of the liposome such that the polymer remains at the liposome surface. Without being bound by any particular theory, it is believed that the polymer(s) will effectively coat the surface of the liposome through a plurality of interactions between the lipid(s) of the liposome and the polymer.

The coupling between the lipids and the polymers may be mediated by any type of interaction—such as a hydrogen bonding interaction, a charge interaction, a hydrophobic interaction, a covalent interaction, a Van Der Waals interaction, or a dipole interaction.

In a preferred embodiment, the interaction is mediated via a covalent interaction.

Preferably, the covalent interaction occurs between one or more groups (eg. functional groups) of the polymer and one or more lipids of the liposome.

One skilled in the art would be able to select suitable groups to achieve the desired interaction between one or more groups (e.g. functional groups) of the polymer and one or more lipids of the liposome.

In one preferred aspect, the covalent interaction occurs between one or more groups of the polymer and one or more functional groups of one or more lipids of the liposome selected from amine, thiol, alcohol, aminoxy, hydrazine, hydrazide, azides, isothiocyanate, aldehydes, ketones, isocyanates, maleimide, halides, tosylates, and esters. Particularly preferred are aminoxy groups and hydrazine groups.

Most preferably, the covalent interaction occurs between one or more aldehyde and/or ketone groups of the polymer and one or more aminoxy functional groups of one or more lipids of the liposome.

Advantageously, the provision of a lipid comprising an aminoxy group allows for simple linking of polymers to the lipid via the aminoxy group. When reacted with a polymer comprising an aldehyde or ketone group, a compound is provided in which the polymer and lipid are linked via an amide group. Such a linkage may be simply prepared in a “one-pot” reaction. This methodology avoids extensive purification procedures by simple dialysis or excess, non-reacted reagents.

Preferably the polymer is selected from mono or bifunctional poly(ethyleneglycol) (“PEG”), poly(vinyl alcohol) (“PVA”); other poly(alkylene oxides) such as poly(propylene glycol) (“PPG”); and poly(oxyethylated polyols) such as poly(oxyethylated glycerol), poly(oxyethylated sorbitol), and poly(oxyethylated glucose), and the like.

As discussed in background teaching US-A-2001/0021763, the polymers can be homopolymers or random or block copolymers and terpolymers based on the monomers of the above polymers, straight chain or branched, or substituted or unsubstituted similar to mPEG and other capped, monofunctional PEGs having a single active site available for attachment to a linker.

Specific examples of suitable additional polymers include poly(oxazoline), poly(acryloylmorpholine) (“PAcM”), and poly(vinylpyrrolidone)(“PVP”). PVP and poly(oxazoline) are well known polymers in the art and their preparation and use in the syntheses described for mPEG should be readily apparent to the skilled artisan. PAcM and its synthesis and use are described in U.S. Pat. No. 5,629,384 and U.S. Pat. No. 5,631,322.

In a preferred embodiment, the polymer is polyethylene glycol (PEG) with a functional aldehyde and/or ketone group or a chemical derivative thereof.

In a preferred embodiment, the polymer has a molecular weight of from 2000 to 1000. In a preferred embodiment, the polyethylene glycol (PEG) has a molecular weight of from 2000 to 1000.

In a preferred embodiment, the polymer has one or more functional groups capable of coupling the one or more lipids. In a preferred embodiment, the polymer has one or two and only one or two functional groups capable of coupling to the one or more lipids.

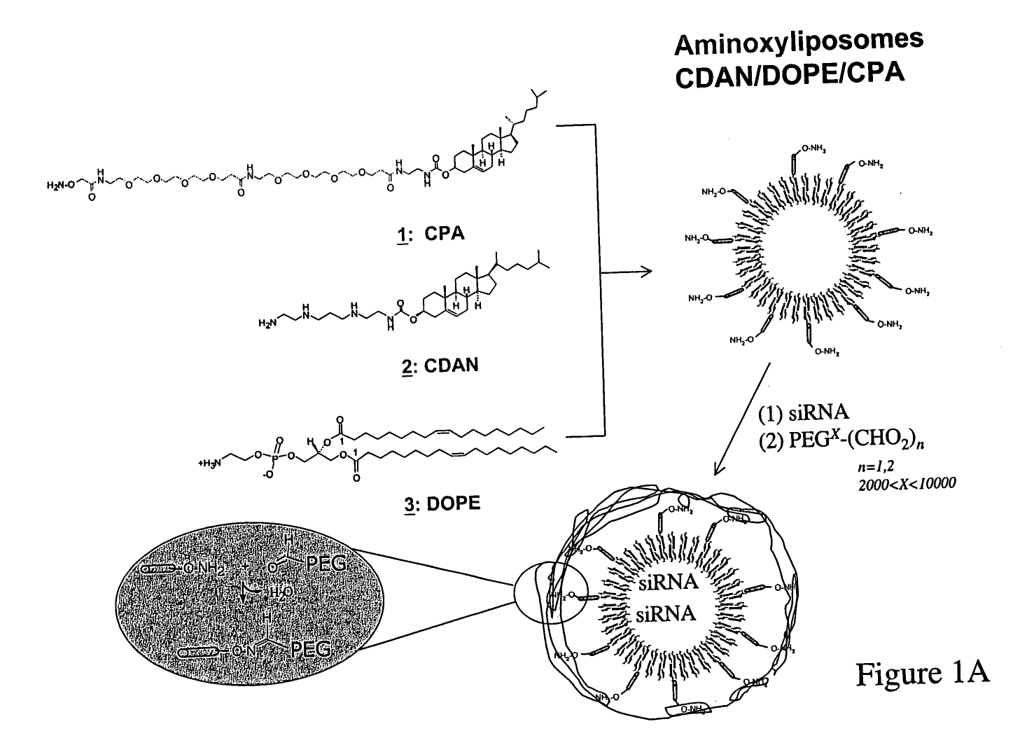

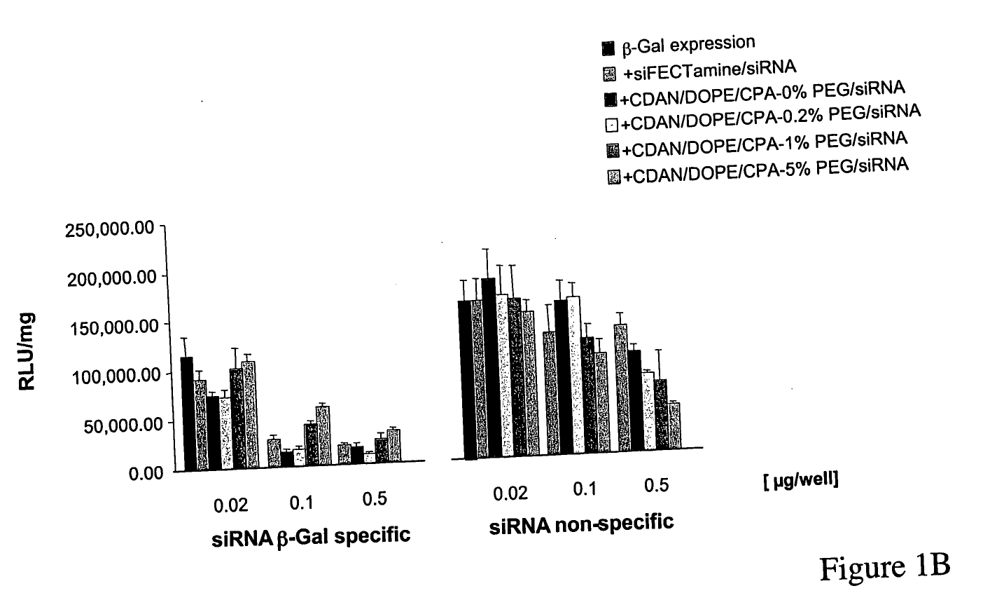

Different sizes of PEG may be used as set forth in FIG. 1 and may include mono-and bis-aldehyde PEG.

Polyethylene glycol (PEG) has previously been used to modify the biophysical properties of drug delivery systems. For example, it can be used to confer serum stability to gene delivery vectors (2-4). This concept has been described in the literature, with the common consensus that the formulation of PEG into gene delivery vehicles dramatically shuts down the transfection efficacy of the gene delivery vector (5-8, 42). Recent investigations into why this phenomenon occurs suggests that PEG formulated non-viral gene delivery systems are equally internalised into cultured cells, but exhibit a dramatically changed intracellular trafficking (9). It appears that these PEG formulated vectors are either not released from endosomes, or that the stealth induced by the PEG does not release the pDNA intracellularly, or that the PEG as a molecule inhibits the intracellular trafficking of the pDNA along the cellular microtubuli system towards the nucleus of the cells. Similar observations have been made with lipoplexes loaded with oligonucleotides (ODN). Song and colleagues observed that unilamellar DODAC/DOPE/PEG-lipid cationic liposome systems with different length of PEG endocytosed like the non-pegylated lipoplexes, but their endosomal release is severely compromised (10). Partially in accordance with these data, Hoekstra and colleagues reported that the inclusion of 10 mol % PEG-phosphatidylethanolamine in ODN lipoplexes inhibited their internalisation in Chinese hamster ovary cells by more than 70%, and that the intracellular fraction remained entrapped in the endosomal/lysosomal pathway. Hoekstra noticed that the procedure of formulation of the PEG into a lipoplex is crucial for the potential of the lipoplex to escape from the endosomes. When liposomes were formed in presence of DSPE-PEG and then loaded with the ODN, the uptake of ODNs was strongly inhibited in a PEG/lipid-concentration dependent manner. Even at lower doses of PEG (1%), complex dissociation was inhibited. In contrast, when DSPE-PEG was incorporated into preformed ODN-lipoplexes, the pegylation is limited to the outer periphery of the ODN-lipoplex, and only such PEGylated complexes appear to display a fertile use in delivery, provided that the PEG-lipids are exchangeable (11).

Based on these results, one would expect that non-viral delivery vectors formulated with siRNA would exhibit the same tendency as observed for ODN and pDNA.

Surprisingly, this is not the case.

Instead, a non-viral delivery vector comprising a liposome, wherein one or more lipids of the liposome are coupled, reversibly or irreversibly, to one or more polymers—such as PEG—and wherein the liposome comprises siRNA dramatically stabilises the liposomes against aggregation without impairing the power of the siRNA to downregulate the target gene.

Accordingly, in a preferred embodiment, the non-viral delivery vector comprises one or more polymers coupled with a liposome comprising one or more aminoxy group containing lipids of the formula (I), preferably CPA and/or a mixture thereof; one or more polycationic lipids selected from the group consisting of DOTMA, DOTAP, DORI-ethers or DORI-esters, (DC-Chol), DOGS, DPPES, and/or CDAN, and/or mixtures thereof; one or more neutral lipids selected from the group consisting of DOPE, POPE, DSPE, DOPC, DPPC, DSPC, DOPG, DPPG DSPG, phosphatidylserines, diphospha tidylglycerols, fatty acid esters, glycerol ester, sphingolipids, cardolipin, cerebrosides; and/or ceramides; and/or mixtures thereof

More preferably, the non-viral delivery vector comprises PEG coupled with a liposome comprising one or more aminoxy group containing lipids of the formula (I), preferably CPA and/or a mixture thereof; one or more polycationic lipids selected from the group consisting of DOTMA, DOTAP, DORI-ethers or DORI-esters, (DC-Chol), DOGS, DPPES, and/or CDAN, and/or mixtures thereof, one or more neutral lipids selected from the group consisting of DOPE, POPE, DSPE, DOPC, DPPC, DSPC, DOPG, DPPG DSPG, phosphatidylserines, diphospha tidylglycerols, fatty acid esters, glycerol ester, sphingolipids, cardolipin, cerebrosides; and/or ceramides; and/or mixtures thereof.

More preferably, the non-viral delivery vector comprises one or more polymers coupled with a liposome comprising CPA, CDAN and DOPE and/or mixtures thereof.

Most preferably, the non-viral delivery vector comprises PEG coupled with a liposome comprising CPA, CDAN and DOPE and/or mixtures thereof.

Non-Viral Delivery Vector

As described herein, the delivery vector is typically made by contacting one or more lipids with the siRNA and any other components to be included in the liposome. The lipids may be part of a pre-formed liposome comprising one or more, preferably, two or more lipid constituents. This final complex may be stored at approximately −80° C. with the addition of 10% sucrose (w/v) until use.

As described herein, the complex may also be freeze-dried in presence or absence of a cryogenic agent such as sucrose, trehalose or lactose without loss of activity and particle integrity for storage purposes.

It is possible to combine the components in any order. Where further components are to be added, they may be added at any stage but preferably together with the siRNA.

Although the non-viral delivery vector may be particularly well suited for pharmaceutical use, they are not limited to that application, and may be designed for food use, agricultural use, for imaging applications, and so forth as described herein.

Agent

In addition to siRNA, the liposome may also include within it one or more agents.

Moreover, and as described herein, the liposome may be coupled, reversibly or irreversibly to one or more agents—such as one more targeting moieties.

The agent may be an organic compound or other chemical. The agent may be a compound, which is obtainable from or produced by any suitable source, whether natural or artificial. The agent may be an amino acid molecule, a polypeptide, or a chemical derivative thereof, or a combination thereof. The agent may even be a polynucleotide molecule—which may be a sense or an anti-sense molecule, or an antibody, for example, a polyclonal antibody, a monoclonal antibody or a monoclonal humanised antibody.

The agent may even be a know drug or compound or an analogue thereof.

The agent may be designed or obtained from a library of compounds, which may comprise peptides, as well as other compounds, such as small organic molecules.

By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetics, a peptide cleaved from a whole protein, or a peptide synthesised synthetically (such as, by way of example, either using a peptide synthesiser or by recombinant techniques or combinations thereof), a recombinant agent, an antibody, a natural or a non-natural agent, a fusion protein or equivalent thereof and mutants, derivatives or combinations thereof.

The agent be an organic compound. For some instances, the organic compounds will comprise two or more hydrocarbyl groups.

The agent may contain halo groups—such as fluoro, chloro, bromo or iodo groups.

The agent may contain one or more of alkyl, alkoxy, alkenyl, alkylene and alkenylene groups—which may be unbranched- or branched-chain.

The agent may exist as stereoisomers and/or geometric isomers—eg. the agent may possess one or more asymmetric and/or geometric centres and so may exist in two or more stereoisomeric and/or geometric forms. The present invention contemplates the use of all the individual stereoisomers and geometric isomers of those agents, and mixtures thereof.

The agent may be any chemical or substance that is desired to be applied, administered or used in a liposome, and may include, but is not limited to pesticides, herbicides, cosmetic agents and perfumes, food supplements including vitamins and minerals, flavourings, and other food additives, imaging agents, dyes, fluorescent markers, radiolabels, plasmids, vectors, viral particles, toxins, catalysts, and so forth.

The agent may include one or more biologically active agents and includes any molecule that acts as a beneficial or therapeutic compound, when administered to an animal, preferably a mammal, more preferably a human, in order to prevent, alleviate or treat a disease. This may include: preventing the disease from occurring in a subject which may be predisposed to the disease but has not yet been diagnosed as having it; inhibiting the disease, ie. arresting its development; or relieving the disease, ie. causing regression of the disease.

Examples of agents include, but are not limited to, anti-inflammatory agents; anti-cancer and anti-tumor agents; anti-microbial and anti-viral agents, including antibiotics; anti-parasitic agents; vasodilators; bronchodilators, anti-allergic and anti-asthmatic agents; peptides, proteins, glycoproteins, and lipoproteins; carbohydrates; receptors; growth factors; hormones and steroids; neurotransmitters; analgesics and anaesthetics; narcotics; catalysts and enzymes; vaccines; genetic material—such as DNA.

The agent may be selected from the group consisting of PEG, sugar, carbohydrate and a ligand.

The sugar may be selected from the group consisting of glucose, mannose, lactose, fructose, maltotriose and maltoheptose ( as shown in FIG. 7).

Chemical Synthesis Methods

The agent and/or the siRNA may be prepared by chemical synthesis techniques.

It will be apparent to those skilled in the art that sensitive functional groups may need to be protected and deprotected during synthesis. This may be achieved by conventional techniques, for example, as described in “Protective Groups in Organic Synthesis” by T W Greene and P G M Wuts, John Wiley and Sons Inc. (1991), and by P. J. Kocienski, in “Protecting Groups”, Georg Thieme Verlag (1994).

It is possible during some of the reactions that any stereocentres present could, under certain conditions, be racemised, for example, if a base is used in a reaction with a substrate having an having an optical centre comprising a base-sensitive group. This is possible during e.g. a guanylation step. It should be possible to circumvent potential problems such as this by choice of reaction sequence, conditions, reagents, protection/deprotection regimes, etc. as is well-known in the art.

The agents and/or the siRNA may be separated and purified by conventional methods.

Separation of diastereomers may be achieved by conventional techniques, e.g. by fractional crystallisation, chromatography or H.P.L.C. of a stereoisomeric mixture of a compound of formula (I) or a suitable salt or derivative thereof. An individual enantiomer of a compound of formula (I) may also be prepared from a corresponding optically pure intermediate or by resolution, such as by H.P.L.C. of the corresponding racemate using a suitable chiral support or by fractional crystallisation of the diastereomeric salts formed by reaction of the corresponding racemate with a suitably optically active acid or base.

The agent and/or the siRNA may be produced using chemical methods to synthesise the agent and/or the siRNA in whole or in part. For example, if the agent comprises a peptide, then the peptide can be synthesised by solid phase techniques, cleaved from the resin, and purified by preparative high performance liquid chromatography (e.g., Creighton (1983) Proteins Structures And Molecular Principles, W H Freeman and Co, New York N.Y.). The composition of the synthetic peptides may be confirmed by amino acid analysis or sequencing (e.g., the Edman degradation procedure; Creighton, supra).

Synthesis of peptide inhibitor agents may be performed using various solid-phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Additionally, the amino acid sequences comprising the agent, may be altered during direct synthesis and/or combined using chemical methods with a sequence from other subunits, or any part thereof, to produce a variant agent.

The term “derivative” or “derivatised” as used herein includes chemical modification of an agent. Illustrative of such chemical modifications would be replacement of hydrogen by a halo group, an alkyl group, an acyl group or an amino group.

The agent may be a modified agent—such as, but not limited to, a chemically modified agent.

The chemical modification of an agent may either enhance or reduce hydrogen bonding interaction, charge interaction, hydrophobic interaction, Van Der Waals interaction or dipole interaction.

Targetted Non-Viral Delivery Vector

In a further aspect, the present invention relates to the targeted delivery of a non-viral delivery vector.

Targeted delivery of the non-viral delivery vector may be achieved by the addition of one or more agents (eg. targeting moieties)—such as peptides and/or other ligands—to the liposome, preferably the surface of the liposome. Advantageously, the agent(s) are coupled to the surface of the liposome via an interaction between the agent(s) and one or more lipids of the liposome that are exposed at the liposome surface.

Advantageously, this may enable delivery of siRNA to specific cells, organs and tissues that can bind the agent(s)—such as the targeting moiety. Typically, the binding between the cells, organs and tissues will be via a specific binding between the cells, organs and/or tissues and the agent.

In a preferred embodiment, the cells, organs and tissues may be or may be derived from liver. As used herein, the term “liver cell” refers to a cell that is located in the liver. Liver cells may include but are not limited to cancerous liver cells, hepatocytes, Kupffer cells, Ito cells, endothelial cells lining the hepatic sinusoids, vascular endothelial cells lining the hepatic blood vessels, and any cells of any origin which happen to reside in the liver (e.g., metastatic cancer cells of ectopic origin).

Preferably, the liver cell is a hepatocye (e.g. HepG2 cells).

Advantageously, the non-viral delivery vectors described herein exhibit a strong accumulation in such cells, organs and tissues.

In another preferred embodiment, the cells, organs and tissues may be or may be derived from the spleen, lung and/or lymph nodes.

As used herein, the term “specific binding” refers to an interaction between one or more cells, organs and/or tissues and an agent(s). This interaction is typically dependent upon the presence of a particular structural feature of the cells, organs and/or tissues—such as an antigenic determinant or epitope—that is recognised by the agent(s), thereby allowing an interaction (eg. binding) to occur.

Preferably, the agent(s) is selected from the group consisting of a sugar, a carbohydrate and/or a ligand.

Preferably, the sugar is selected from the group consisting of glucose, mannose, lactose, fructose, maltotriose, maltoheptose.

The agent may even be a ligand.

Many different ligands may be employed, depending upon the site targeted for liposome delivery.

The ligand may be designed or obtained from a library of compounds, which may comprise peptides, as well as other compounds, such as small organic molecules.

By way of example, the ligand may be a natural substance, a biological macromolecule, or an extract made from biological materials such as bacteria, fungi, or animal (particularly mammalian) cells or tissues, an organic or an inorganic molecule, a synthetic ligand, a semi-synthetic ligand, a structural or functional mimetic, a peptide, a peptidomimetic, a derivatised ligand, a peptide cleaved from a whole protein, or a peptide synthesised synthetically (such as, by way of example, either using a peptide synthesiser or by recombinant techniques or combinations thereof, a recombinant ligand, an antibody, a natural or a non-natural ligand, a fusion protein or equivalent thereof and mutants, derivatives or combinations thereof).

Preferably, the ligand is an antibody.

As used herein, the term “antibody” refers to complete antibodies, bi-specific antibodies or antibody fragments, and includes Fv, ScFv, Fab′ and F(ab′)₂, monoclonal and polyclonal antibodies, engineered antibodies including chimeric, CDR-grafted and humanised antibodies, and artificially selected antibodies produced using phage display or alternative techniques.

A chimeric antibody refers to a genetically engineered fusion of parts of a mouse antibody with parts of a human antibody. Generally, chimeric antibodies contain approximately 33% mouse protein and 67% human protein.

Humanised antibodies may be obtained by replacing the constant region of a mouse antibody with human protein, but by also replacing portions of the antibody's variable region with human protein. Generally humanised antibodies are 5-10% mouse and 90-95% human.

A more sophisticated approach to humanised antibodies involves not only providing human-derived constant regions, but also modifying the variable regions as well. This allows the antibodies to be reshaped as closely as possible to the human form. This approach has been reported in, for example, Cancer Res (1993) 53:851-856, Nature (1988) 332:323-327, Science (1988) 239:1534-1536, Proc Natl Acad Sci USA (1991) 88:4181-4185 and J Immunol (1992) 148:1149-1154.

Preparation of antibodies is performed using standard laboratory techniques Antibodies may be obtained from animal serum, or, in the case of monoclonal antibodies or fragments thereof, produced in cell culture. Recombinant DNA technology may be used to produce the antibodies according to established procedure, in bacterial or preferably mammalian cell culture. The selected cell culture system preferably secretes the antibody product.

The foregoing, and other, techniques are discussed in, for example, Kohler and Milstein, (1975) Nature 256:495-497; U.S. Pat. No. 4,376,110; Harlow and Lane, Antibodies: a Laboratory Manual, (1988) Cold Spring Harbor. Techniques for the preparation of recombinant antibody molecules are described in the above references and also in, for example, EP 0623679; EP 0368684 and EP 0436597.

Antibodies may be selected and generated using phage display technology. Methods for the construction of bacteriophage antibody display libraries and lambda phage expression libraries are well known in the art (eg. Kang et al. (1991) Proc. Natl. Acad. Sci. U.S.A., 88: 4363 and Clackson et al. (1991) Nature, 352: 624).

Advantageously, when the ligand is an antibody, the lipoplex coupled antibody demonstrates substantially all of it activity.

Preferably, the ligand is a receptor—such as a receptor that is or is derived from a RGD peptide (integrin receptor), a folate receptor and/or a transferrin receptor.

Pharmaceutical Salt

The non-viral delivery vectors may be administered in the form of a pharmaceutically acceptable salt.

Pharmaceutically-acceptable salts are well known to those skilled in the art, and for example, include those mentioned by Berge et al, in J. Pharm. Sci., 66, 1-19 (1977). Suitable acid addition salts are formed from acids which form non-toxic salts and include the hydrochloride, hydrobromide, hydroiodide, nitrate, sulphate, bisulphate, phosphate, hydrogenphosphate, acetate, trifluoroacetate, gluconate, lactate, salicylate, citrate, tartrate, ascorbate, succinate, maleate, fumarate, gluconate, formate, benzoate, methanesulphonate, ethanesulphonate, benzenesulphonate and p-toluenesulphonate salts.

When one or more acidic moieties are present, suitable pharmaceutically acceptable base addition salts can be formed from bases which form non-toxic salts and include the aluminium, calcium, lithium, magnesium, potassium, sodium, zinc, and pharmaceutically-active amines such as diethanolamine, salts.

Pharmaceutically Active Salt

The non-viral delivery vectors may be administered as a pharmaceutically acceptable salt. Typically, a pharmaceutically acceptable salt may be readily prepared by using a desired acid or base, as appropriate. The salt may precipitate from solution and be collected by filtration or may be recovered by evaporation of the solvent.

Pharmaceutical Compositions

Pharmaceutical compositions of the present invention may comprise a therapeutically effective amount of the non-viral delivery vectors.

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

If the agent is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions may be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or the pharmaceutical compositions can be injected parenterally, for example, intravenously, intramuscularly or subcutaneously. For parenteral administration, the compositions may be best used in the form of a sterile aqueous solution which may contain other substances, for example, enough salts or monosaccharides to make the solution isotonic with blood. For buccal or sublingual administration the compositions may be administered in the form of tablets or lozenges which can be formulated in a conventional manner.

The non-viral delivery vectors may be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

The pharmaceutical composition comprising the non-viral delivery vectors may also be used in combination with conventional treatments for the disease of interest.

Administration

The non-viral delivery vectors may be administered alone but will generally be administered as a pharmaceutical composition—eg. when the vectors are in admixture with a suitable pharmaceutical excipient, diluent or carrier selected with regard to the intended route of administration and standard pharmaceutical practice.

For example, the non-viral delivery vectors may be administered in the form of tablets, capsules, ovules, elixirs, solutions or suspensions, which may contain flavouring or colouring agents, for immediate-, delayed-, modified-, sustained-, pulsed- or controlled-release applications.

If the pharmaceutical is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents—such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) may include, but are not limited to, one or more of oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

Dose Levels

Typically, a physician will determine the actual dosage which will be most suitable for an individual subject.

The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Formulation

The non-viral delivery vectors may be formulated into a pharmaceutical composition, such as by mixing with one or more of a suitable carrier, diluent or excipient, by using techniques that are known in the art.

Diseases

Aspects of the present invention may be used for the treatment and/or prevention of diseases such as those listed in WO-A-98/09985.

For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; diseases associated with viruses and/or other intracellular pathogens; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue. Specific cancer related disorders include but not limited to: solid tumours; blood born tumours such as leukemias; tumor metastasis; benign tumours, for example hemangiomas, acoustic neuromas, neurofibromas, trachomas, and pyogenic granulomas; rheumatoid arthritis; psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; wound granulation; corornay collaterals; cerebral collaterals; arteriovenous malformations; ischeniic limb angiogenesis; neovascular glaucoma; retrolental fibroplasia; diabetic neovascularization; heliobacter related diseases, fractures, vasculogenesis, hematopoiesis, ovulation, menstruation and placentation.

Aspects of the present invention may be used for the treatment and/or prevention of diabetes—such as diabetes I and II.

Aspects of the present invention may also be used for the treatment and/or prevention of cancer—such as acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, stomach cancer, testicular cancer, thyroid cancer, urinary system cancer, uterine sarcoma, vaginal cancer, vascular system, Waldenstrom's macroglobulinemia and Wilms' tumor.

In a preferred embodiment, the disease is a disease, disorder or condition that is or is associated with liver disease and/or liver damage.

Liver damage may be associated with exposure to alcohol, hepatotoxic drugs and combinations thereof. For example, damaging agents may include anti-convulsants, phenytoin, carbamazepine and phenobarbital, recreations drugs—such as ecstasy (3,4-methylenedioxymethamphetamine), antituberculosis agents and chemotherapeutic agents—such as isoniazid and rifampicin.

Liver damage may also be associated with infectious agents—such as bacterial, parasitic, fungal and viral infections. For example, liver damage may result from Aspergillus fungal infections, Schistosoma parasitic infections and a variety of viral infections—such as adenovirus, retrovirus, adeno-associated virus (AAV), hepatitis virus A, hepatitis virus B, hepatitis virus C, hepatitis virus E, herpes simplex virus (HSV), Epstein-Barr virus (EBV) and paramyxovirus infections.

Liver diseases may include, but are not limited to, acute hepatitis, fulminant hepatitis, chronic hepatitis, hepatic cirrhosis, fatty liver, alcoholic hepatopathy, drug induced hepatopathy (drug addiction hepatitis), congestive hepatitis, autoimmune hepatitis, primary biliary cirrhosis, hepatic porphyria, pericholangitis, sclerosing cholangitis, hepatic fibrosis and chronic active hepatitis.

Uses

The non-viral delivery vectors described herein may be used to efficiently transfect cells—such as eukaryotic cells, in particular mammalian cells, with siRNA.

The non-viral delivery vectors described herein may be used to efficiently transect the liver.

The non-viral delivery vectors may be used in a variety of siRNA delivery applications—such as gene therapy, DNA vaccine delivery and in vitro transfection studies.

The non-viral delivery vectors may be used in a variety of siRNA delivery applications—such as gene therapy, DNA vaccine delivery and in vitro transfection studies—of the liver.

The non-viral delivery vectors may also be used to administer therapeutic genes to a patient suffering from a disease.

Variants/Homologues/Derivatives

The present invention also encompasses the use of homologues and fragments.

Here, the term “homologue” means an entity having a certain homology with the nucleotide sequences described herein. Here, the term “homology” can be equated with “identity”.

In the present context, a homologous sequence is taken to include a nucleotide sequence, which may be at least 75, 85 or 90% identical, preferably at least 95 or 98% identical to a subject sequence. Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons may be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A tool, called BLAST 2 Sequences is also available for comparing protein and nucleotide sequence (see FEMS Microbiol Lett 1999 174(2): 247-50; FEMS Microbiol Lett 1999 177(1): 187-8).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in to enhance the in vivo activity or life span of nucleotide sequences useful in the present invention.

The present invention may also involve the use of nucleotide sequences that are complementary to the sequences described herein, or any derivative, fragment or derivative thereof.

Alleles of the sequences are also included. An “allele” or “allelic sequence” is an alternative form of the sequence encoding a protein. Alleles result from a mutation, ie., a change in the nucleic acid sequence, and generally produce altered mRNAs or polypeptides whose structure or function may or may not be altered. Preferably, function is not altered. Any given gene may have none, one or many allelic forms. Common mutational changes which give rise to alleles are generally ascribed to deletions, additions or substitutions of amino acids. Each of these types of changes may occur alone, or in combination with the others, one or more times in a given sequence.

The term “allele” also includes genetic polymorphisms—such as SNPs (single nucleotide polymorphisms).

The term “fragment” in relation to a nucleotide sequence includes any substitution of, variation of, modification of, replacement of, deletion of or addition of one (or more) nucleotide sequences from or to the sequence providing the resultant protein has biological activity, preferably being at least as biologically active as the protein encoded by the full length nucleotide sequence. The fragment may comprises 50, 60, 70, 80, 90, 95, 96, 97, 98 or 99% of the full length nucleotide sequence.

In a further aspect, there is also provided a method for delivering siRNA to a cell or the environment of a cell, comprising the step of providing to the environment of a cell, tissue or organ (eg. the liver) the non-viral delivery vector or the targeted delivery vector described herein.

General Recombinant DNA Methodology Techniques

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press.

The invention will now be further described by way of Examples, which are meant to serve to assist one of ordinary skill in the art in carrying out the invention and are not intended in any way to limit the scope of the invention.

### EXAMPLES

Materials & Methods

Thin Layer Chromatography

Thin layer chromatography (TLC) was performed on pre-coated Merck-Kieselgel 60 F₂₅₄ aluminium backed plated and revealed with ultraviolet light, iodine, acidic ammonium molybdate (IV), acidic ethanolic vanillin, or other agents as appropriate. Flash column chromatography was accomplished on Merck-Kieselgel 60 (230-400 mesh). Mass spectra were recorded using Brucker Esquire 3000, VG-7070B or JEOL SX-102 instruments. ¹H and ¹³C NMR resonances were recorded on Advance Brucker 400 Ultrashield™ machine using residual isotopic solvent as an internal reference (s=singlet, d=doublet, t=triplet, q=quartet, quin=quintet, br=broad singlet).

siRNA

Anti-β-gal siRNA-1 (5′-CUA CAC AAA UCA GCG AUU UUU-3′) which was used throughout this study to evaluate the down-regulation of the lacZ gene product was purchased from Dharmacon (USA) and stored as a 20 μM solution as indicated by the manufacturer. The non-specific siRNA sequence (5′-UAG CGA CUA AAC ACA UCA AUU-3′) was obtained from Dharmacon (USA) stored at 20 μM as indicated by the manufacturer. The 3′-FITC labelled anti-GFP siRNA sequence (5′ GGC UAC GUC CAG GAG CGC ACC-3′) was obtained from Qiagen GmbH (Hilden, Germany). Sequences may be derived from an algorithm that verifies off-target down-regulation (Kumiko Ui-Tei et al, Nucl. Acids Res. 2004, Vol 32, No. 3, p. 936-48). One such sequence derived from this algorithm is a anti-beta-Gal sequence targeting the lacZ gene 648-670 upstream of the ATG start-codon (5′-GCA UAA ACC GAC UAC ACA AAU-3′). This sequence is different from the Dharmacon sequence in lacking potential off-target down-regulation of, for example, the human genes TIMM8A (translocase of inner mitochondrial membrane 8 homolog A) and NM_{—}001132.1 (AFG3 ATPase family gene 3) and others.

Preparation of Liposomes/Lipoplexes

All CDAN/DOPE/aminoxy lipids were prepared by pipetting the appropriate amount of stock solutions of CDAN, DOPE and aminoxylipid (CPA), respectively, into a round bottomed flask pre-treated with nitric acid (HNO₃, pure; 10 mins) and dimethylsilyldichlorid (Sigma, UK10; mins), evaporating the solvent, and hydrating the dry lipid film with water (milliQ, 18 Ω) under heavy vortexing, to generate multilamellar liposomes, pH ∞3.5-4, at 3 mg/mL total lipid. Unilamellar liposomes were produced by sonicating the multilamellar liposomes for 30 mins in a Sonomatic water bath (Longford Ultrasonics, UK). By extensively sonicating the liposomes, the solution becomes totally transparent, and the liposome size cannot be determined anymore by PCS, which is an indication that the liposome size was smaller than 30 nm. A solution of siRNA in water (0.28 mg/mL) was added drop-wise to these CDAN/DOPE/CPA liposomes (3 mg/mL) under heavy vortexing at a final siRNA concentration of 0.1 mg/mL. The resulting siRNA lipoplex typically measured 30-50 nm diameter as determined by PCS. A solution of polyethyleneglycol-α/γ-bisaldehyde (Mw 2000, or 3400, respectively; NEKTAR, USA) at 1 mg/mL was prepared and the appropriate amount added to the lipoplex under vortexing to give covalently surface-pegylated siRNA-lipoplexes with differential amounts of PEG (0.1-5%, m/m total lipid). Half of the volume was evaporated under reduced pressure and compensated by addition of PBS prior to injection into animals.

Cell Cultures

HeLa or IGROV-1 cells were seeded in 48-well plate at 40,000 cells/well 24 h before the experiment in growth medium (DMEM/10% FCS/penicillin/streptomycin) and cultured at 37° C. (10% CO₂). Prior to transfection, the medium was replaced with fresh growth medium. OptiMEM-I/DMEM were purchased from Invitrogen (UK).

In Vivo Transfection

A. Hydrodynamics model. Balb/C mice of 20-25 g body weight were injected with siRNA-containing lipoplexes (200 uL, 0.1 mg/mL siRNA) via tail vein injection or via injection into the intra-peritoneal cavity, and left for 8 h or 24 h, respectively. After this respective resting phase, the animals were injected with 1 μg pUMVC1 (7528 Bp, University of Michigan Vector Core, coding for the lacZ gene under the CMV promotor; http://www.med.umich.edu/vcore/Plasmids/) in 2.5 mL PBS within 10 seconds, and left for 24 h before dissecting the liver and assaying for beta-galactosidase activity using a standard ELISA test (ROCHE). The resulting relative light units were standardised by dividing through the total cellular protein content as measured by the BCA assay (Pierce).

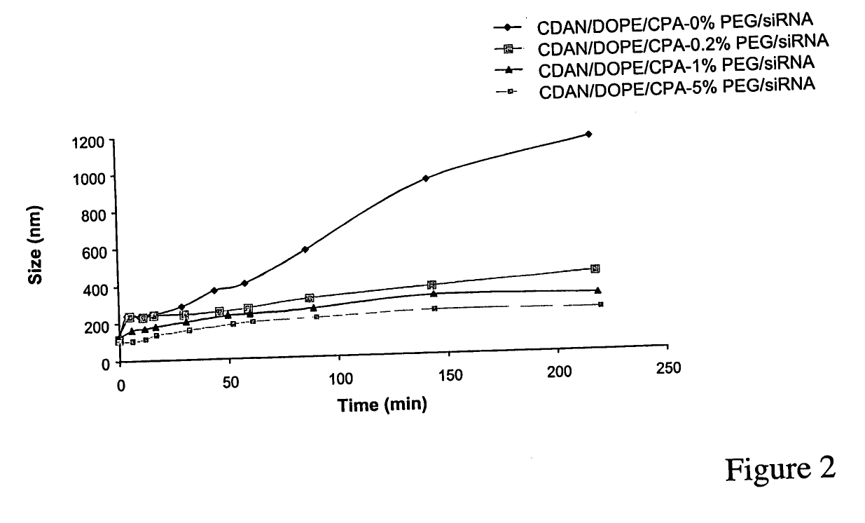

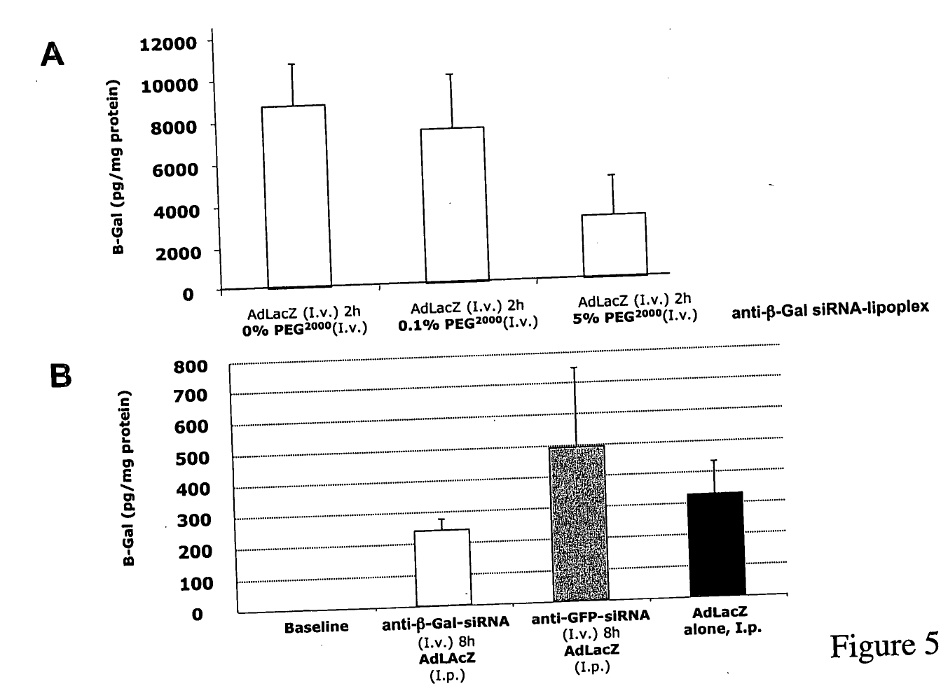

B. Recombinant adenovirus(lacZ) model. AdRSVBGal was purchased from Transgene (Strasbourg, France). It is a type-5 adenovirus with the majority of the E3 region deleted. The virus was titrated by injecting different amounts of the stock into Balb/C mice, and a titre chose where approximately 10,000 RLU/mg protein liver expression was obtained. Typically, 15 μL of virus stock was diluted in PBS (200 μL) and injected into the tail veins or the intra-peritoneal cavity, respectively, of Balb/C mice. Two protocols were followed: 1. Injection of the AdRSVβGal 2 h prior to injection of siRNA-lipoplex (FIG. 5A) and 2. Tail vein-Injection of 200 μL siRNA-lipoplex (0.1 mg/mL siRNA) per animal 8 h prior to injection of the AdRSVβGal (intra-peritoneal) (FIG. 5B). All beta-galactosidase ELISA assays were carried out 24 h post-viral injection.

Transfection

The β-Gal reporter gene (pUMVCl-β-Gal, 7528 Bp) was transfected with PRIMOfectTM (IC-Vec Ltd., UK) according to the manufacturer's instructions. Typically, 0.1 μg (HeLa) or 0.25 μg (IGROV-1) pDNA were transfected per 48-well. The total nucleic-acid:lipid ratio was 1:12 (w/w) as recommended in the instruction manual. After a pDNA transfection time of 3 h, transfection medium was replaced with fresh growth medium (150 μL) after which LsiR siFection experiments were performed using LsiR particles prepared in fresh OptiMEM (final volume 100 μL) just prior to siFection.

All siFections described in this work were carried out on 48-well plates. For this purpose, the siRNA (0.1 μg) was diluted with fresh OptiMEM to a final volume of 100 μL. 4.35 μL CDAN/DOPE (0.3 mg/mL) was added under vortexing (to give a lipid/siRNA ratio 13:1 [w/w]) and the LsiR lipoplex allowed to stand for 5 minutes. Finally the LsiR mixture was then introduced to the appropriate well of a given 48-well plate containing cells in complete growth medium (including FCS/antibiotics) (150 μL) and incubated at 37° C., 10% CO₂ for 3 h. Medium was then replaced with fresh growth medium and cells were incubated for 16-72 h before β-Gal reporter assay (Roche, UK).

### Example 1

PEGylated siRNA-Lipoplexes

Referring to FIG. 1A, DOPE (140 μL, 10.68 mg/mL in CHCl₃), CDAN·3HCl (271 μL, 4 mg/mL in CHCl₃) and CPA (100 μL, 4.4 mg/mL in CHCl₃) were pipetted into a round bottled flask (5 mL) pretreated with nitric acid and dimethyldichlorosilane, and the solvent removed under reduced pressure to form a lipid film, which was hydrated by the addition of water (milliQ, lmL) under vortexing. Subsequently, the multilamellar liposome formulation was sonicated in a sonomatic® water bath (Longford Ultrasonics) for 30 mins to give small unilamellar vesicles (SUV). 250 μL of these liposomes were pipetted into a 5 mL falcon tube, and a solution of siRNA (0.28 mg/mL) added drop wise under heavy vortexing, followed by the addition of polyethylene glycol-bisaldehyde (Mw 3400, 7.8 μL, 10 mg/mL; 5% PEG/total lipid). The sample was left standing for 15 mins/RT before adding PBS (483 μL). After leaving the sample for 16 h/RT, the volume was reduced to 750 μL to give an siRNA-lipoplex (LsiR) of 0.1 mg/mL siRNA.

Referring to FIG. 1B, pUMVC1-β-Gal was transfected with PRIMOfectTM (IC-Vec Ltd., UK) according to the manufacturer's instructions. Typically, 0.1 μg (HeLa) or 0.25 μg (IGROV-1) pDNA were transfected per 48-well. After a pDNA transfection time of 3 h, transfection medium was replaced with fresh growth medium (150 μL). LsiR complexes with different amounts of PEG (0-5%) were generated as described under A. For each experiment, 3 doses of siRNA were chosen (0.02/0.1/0.5 μg siRNA/well; 5/30/150 nM) and diluted with OptiMEM to give a final volume of 100 μL that were added to each well (48 well plate) containing 150 μL fresh growth medium and cells were incubated for 16-72 h before β-Gal reporter assay (Roche, UK).

### Example 2

Stability of Pegylated siRNA-Lipoplexes

The stability of pegylated (Mw 2000) siRNA-lipoplexes in 80% serum (FCS) was inventiagted. Surface-pegylated LsiR complexes were generated as described in Example 1 and FIG. 1 and incubated with FCS for different times before measuring the particle sizes by photon correlation spectroscopy (PCS). The results are shown In FIG. 2. With increasing degree of PEG, the particle size does not increase over the investigated time scale.

Advantageously, PEGylated siRNA-lipoplexes generated by post-coupling PEG to an siRNA loaded lipoplex exhibit serum stability with increasing amounts of PEG coupled to the surface.

### Example 3

Tissue Distribution Studies

The liposome formulation was labeled with the lipid [4-¹⁴C]cholesterol (Amersham Biosciences) at final molar ratios of CDAN/DOPE/CPA700/[4-¹⁴C]cholesterol 39.95:50:10:0.05. siRNA lipoplexes were made at a ratio liposome/siRNA 13:1 (w/w) by addition of the siRNA into the liposomes under vortexing. Samples were concentrated to half of the total volume and compensated to the original volume by adding PBS. 200 μl (0.1 mg/mL siRNA) of these complexes were injected into the lateral tail vein of each mouse weighing approximately 30 g. Radioactivity was adjusted to approximately 0.035 μCI per animal.

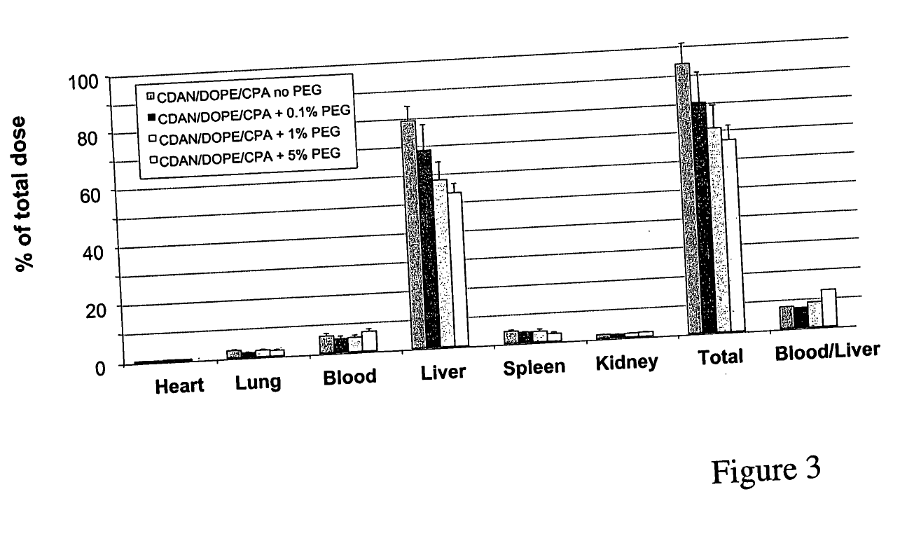

After 1 hour the mice were anaesthetised and blood was obtained by cardiac puncture and immediately mixed with 15 U of heparin. The blood concentration of the liposomes was calculated assuming that the total blood weight was 6% of the body weight. After cervical dislocation, liver, spleen, kidney, lung, and heart were dissected and weighed. Organs were homogenized in PBS at a concentration of 5 mL PBS/g of organ. Aliquots of 200 μL of each organ and 100 μL of blood were solubilized with Solvable at 60° C. for 1 hour. Samples were then treated with 0.1 mL of EDTA (0.1 M) followed by 0.3 mL-0.5 ml 30% hydrogen peroxide in 0.1 mL aliquots. After 15-30 minutes at room temperature, samples were incubated one more time at 60° C. for 1 hour. Then, 10 mL of Ultima GoldTM was added to each vial and radioactivity was determined using a liquid scintillation counter. The results shown in FIG. 3 are expressed as a percentage of injected dose per organ (%ID).

PEGylated siRNA-lipoplexes generated by post-coupling PEG to an siRNA loaded lipoplex exhibit a pharmacokinetics profile different from the non-pegylated analogue, gradually decreasing the amount detected in the liver with increasing amounts of PEG.

### Example 4

Down-Regulation of the lacZ Gene in the Liver

200 μL LsiR pegylated with PEG²⁰⁰⁰(CHO)₂ (0.1%) was injected intravenously to Balb/C mice and the animals allowed to rest for 8 hours, before injecting 1 μg pUMVC1-β-Gal in 2.5 mL PBS within 10 seconds (hyperdynamic injection). The animals were sacrificed 24 h post-pDNA injection and assayed for beta-galactosidase expression by ELISA. Note that the pDNA only and the pDNA plus the LsiR(GFP control) yield identical expression levels of β-Gal, whereas the LsiR(anti-B-Gal) mediates more than 80% down-regulation of the β-Gal protein.

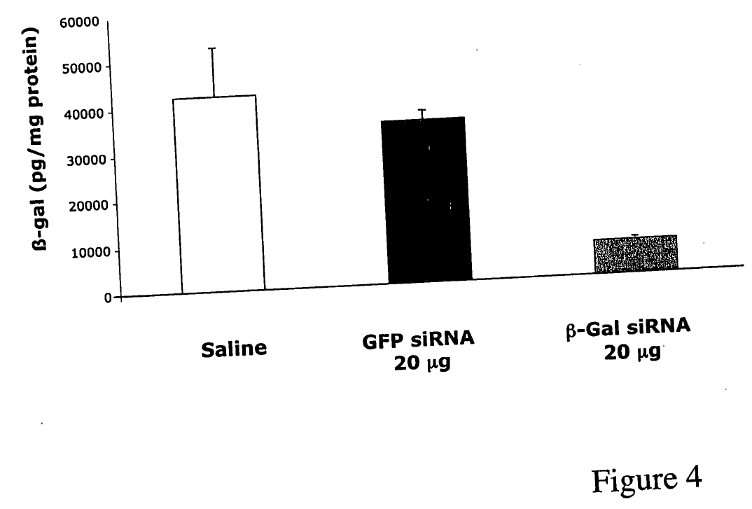

The results are shown in FIG. 4.

Surprisingly, the down-regulation of the lacZ gene that was introduced into the liver of female Balb/C mice by hydrodynamic injection of 1 μg pDNA (in 2 ml PBS) reached more than 80% after systemic delivery of 20 μg siRNA-lipoplex (PEG 0.1%) 8 or 24 hours post-hydrodynamic injection.

### Example 5

Down-regulation of adeno-virally expressed J3-Gal protein with pegylated LsiR lipoplexes.

To study the effect of the level of pegylation on down-regulation of adeno-virally expressed β-Gal protein, AdRSVβGal was injected via tail vein injection (200 μL total volume) and the animals left for 2 h before injecting LsiR(anti-B-Gal) pegylated with 0/0.1/5% PEG²⁰⁰⁰(CHO)₂ respectively. The results are shown in FIG. 5A.

To study the effect of the injection route of the adenovirus, AdRSVBGal was injected into the intra-peritoneal cavity (200 μL total volume) 8 h after intravenous injection of LsiR lipoplexes pegylated with PEG³⁴⁰⁰(CHO)₂ (5%). Due to the different route of injection of the AV, the total levels of β-Gal protein were reduced to 10% of the levels obtained by the i.v. route.

The results are shown in FIG. 5B.

Surprisingly, male Balb/C mice that were infected with a given dose of a lacZ-Adenovirus and 2 h post-viral infection obtained 20 μg siRNA-lipoplex (PEG 0%/0.1%/5%) via tail vain injection showed maximum downregulation (>70%) of the highest PEGylated siRNA-lipoplex (5% PEG).

### Example 6

(A)

HPLC Analysis

HPLC analysis of CDAN/CPA/DOPE (20:30:50, m/m/m) liposomes was performed. 30 μL of liposomes (3 mg/mL) were diluted with 70 μL water, and 90 μL of this solution injected into an HPLC. The peaks were analyzed by an evaporating light scattering detector (ELS). The results are shown in FIG. 6A.

Antibody coupling onto the surface of CDAN/CPA/DOPE (20:30:50, m/m/m) was also investigated. 2 mg of Rabbit IgG (Sigma) were dissolved in lmL NaOAc (20 mM), NaCl (0.15 M) pH 5.9; In a separate tube, lmL of fresh H₅IO₆ were prepared and the two tubes combined and left at room temperature for 1.5 h. The reaction was quenched by the addition of 0.5 mL ethyleneglycol prior to transferring the whole reaction mixture into a dialysis tube (Spectrum Labs, USA; MWCO 12000-14000) and dialyzed against 0.1 M K₂HPO₄/0.1% TritonX for 16 h. The solution was recovered and the IgG^{OX} concentration determined by the BCA assay. 80 μL CDAN/CPA/DOPE (20:30:50, m/m/m; 2 mg/mL) and 100 μL oxidized IgG^{OX} (0.94 mg/mL) were incubated at 37oC./16 h and 90 μL injected into the HPLC for analysis. The results are shown in FIG. 6A. The CPA peak at rt=27 mins decreased by 48% compared to the control where non-oxidized IgG was incubated with liposomes. A new peak at rt=36 mins is observed with a strong absorbance at λ=280 nm indicative for proteins, which was isolated and analyzed on SDS page and found to be IgG with about 20 copies of CPA covalently bound through the oxidized Fc-carbohydrate units via an oxime bond.

Double incubation of liposomes with (i) PEG²⁰⁰⁰(CHO)₂ followed by (ii) IgG^{OX} was also performed. The results are shown in FIG. 6C. Surprisingly, the high reactivity of the aminoxy functional group of the CPA lipid allows the incubation of the CDAN/CPA/DOPE (20:30:50, m/m/m) liposomes with PEG²⁰⁰⁰(CHO)₂ (1%) first to generate a serum protected liposomes which can be further reacted with the oxidized IgG^{OX} to conjugate the antibody covalently to the surface of the pegylated liposome (FIG. 6C(B)). For this purpose, 30 μL CDAN/CPA/DOPE (20:30:50, m/m/m) liposomes were incubated with 2 μL PEG²⁰⁰⁰(CHO)₂ (10⁻⁸ mol) for 15 mins/RT before addition of 40, 60, 80 or 100 μL oxidized IgG^{OX} (0.94 mg/mL), respectively (FIG. 6C(B), 1-4). The reaction mixture was incubated for 16 h, and 90 μL injected into HPLC for analyses (FIG. 6C(A and B)). The CPA signal at rt=27 mins decreases gradually with increasing amounts of IgG^{OX} (bottom panel) despite the prior incubation with PEG(CHO)₂, indicating that the remaining free aminoxy groups of the CPA lipid are still reactive to covalently conjugate the antibody to the surface of the pegylated liposomes. Note that the HPLC signal at rt=35 min exhibits a strong absorbance at k=280 nm indicative for proteins.

LsiR lipoplexes made from siRNA and CDAN/DOPE/CPA (40/50/10; m/m/m) liposomes pegylated at 0.1-1% total lipid (molar ratio in the lipoplex) can be incubated with an oxidized IgG antibody at acidic pH, resulting in the covalent coupling of the antibody through its partially oxidized carbohydrate units to the CPA lipid as demonstrated by HPLC analyses of aminoxy liposomes before incubation with oxidized IgG (FIG. 6a) and after incubation with oxidized IgG (FIG. 6b).

(B)

(i) Liposomes and IgG Preparation

(CDAN/DOPE/CPA): 164 μL of DOPE (9.05 mg/ml, 744 g/mol), 279 μL CDAN·3HCl (3.88 mg/mL, 680 g/mol) and 107 μL CPA (4 mg/mL, 1075 g/mol) were mixed in a 5 mL round bottomed flask and the solvent was evaporated at approximately 30° C. to form a dry lipid film. By adding lmL water and vortexing for 1 minute multilamellar liposomes were generated. The liposome sample was sonicated for 20 minutes to generat small unilamellar vesicles of <100 nm size.

CDAN/DOPE/CPA/DSPE^{rhod}: 159 μL of DOPE (9.05 mg/ml, 744 g/mol), 277 μL CDAN·3HCl (3.88 mg/mL, 680 g/mol), 45 μL CPA (8 mg/mL, 1075 g/mol) and 25.8 μL DSPE-rhodamine (2 mg/mL, 1301 g/mol) were mixed in a 5 mL round bottomed flask. The solvent was evaporated at approximately 30° C. to form a dry lipid film. By adding lmL water and vortexing for lminute multi lamellar liposomes were generated.The liposome sample was sonicated for 20 minutes to generat small unilamellar vesicles of <100 nm size.

Oxidation of IG: (a) 260 μL of the IgG stock (0.38 mg/mL) and 260 μL periodic acid (20 mM in water) were combined and left for 30 minutes at room temperature in the dark. The sample was then desalted in a NAP-5-column. After the oxidation different IgG dilutions were prepared (0 μL-170 μL of the oxidized IgG filled up with water to a total volume of 170 μL) and mixed with 30 μL liposome (3 mg/mL). A sample of 40 μg protein was analysed on SDS page gel (FIG. 6d, A1, lane 2).

(b) 250 μL of the IgG stock (0.38 mg/mL) and 250 μL periodic acid (20 mM in water) were combined and left for 1 hour at room temperature in the dark. The sample was then dialyzed against sodium acetate buffer (20 mM sodium acetate and 150 mM NaCi, pH 5.5) for 2 hours. After the oxidation different IgG dilutions were prepared (0 μL-170 μL of the oxidized IgG filled up with water to a total volume of 170 μL) and mixed with 30 μL liposome. A sample of 40 μg protein was analysed on SDS page gel (FIG. 6d, A1, lane 3).

(c) 250 L of the IgG stock (0.38 mg/mL) and 250 μL periodic acid (20 mM in water) were combined and left for 2 hours at room temperature in the dark. The sample was then dialyzed against sodium acetate buffer (20 mM sodium acetate and 150 mM NaCl, pH 5.5) for 2 hours. After the oxidation different IgG dilutions were prepared (1 μL-70 μL of the oxidized IgG filled up with water to a total volume of 70 μL) and mixed with 30 μL liposome. A sample of 40 μg protein was analysed on SDS page gel (FIG. 6d, A1, lane 4).

IgG^{OX} fluorescence labelling: 70 μL (0.2 mg/mL) antibody was fluorescently labeled by incubating with 50 μL NHS-FITC (lOmg/mL, DMSO). Purification was achieved in slide-A-Lyzer mini dialysis units, 10,000 MWCO. Determination of the protein concentration was done by the BCA test and found to be 0.11 mg/mL.

ELISA: Into each well of the NUNC-Immuno plate 5 μL HFN (lmg/mL) and 40 μL TRIS buffer (NaCl 0.25 M, TRIS 0.02 M. pH 7.6) were added and the plate was incubated at room temperature for 30 minutes. After that the plate was washed three times with washing buffer (Tris 0.02M, NaCl 0.5 M, triton 0.5%, pH 7.6). By adding 3 μL BSA (lmg/mL) and 40 μL Tris buffer to each well the wells were blocked. Then the plate was incubated for 1 hour at 37° C. After this the plate was washed and 75 μL of different dilutions between 0 and 60 pmol oxidized IgG (0.18 mg/mL), or IgG^{OX}-coupled lipoplexes (0.11 mg/mL protein) added. The plate was incubated for 1 hour at 37° C. and washed afterwards. Next 50 μL of a sheep anti-mouse antibody coupled to horseradish peroxidase (diluted 1:5000) were added into each well. The plate was incubated for another hour at 37° C. and washed. In the end 50 μL of a SIGMA FASTTMOPD substrate were added to each well and the absorbance at 405 nm was measured in a plate reader.

(ii) Formation of Lipoplexes

Lipoplex 1; CDAN/DOPE/CPA and Cy3-GFP labeled siRNA. 100 L of the CDAN/DOPE/CPA liposome stock (3 mg/mL) were mixed with 125 μL water. While vortexing the mixture, 75 μL siRNA Cy3-GFP (0.4 mg/mL) were added slowly to the solution. Then 53 μL PEG²⁰⁰⁰(CHO)₂ (0.135 mg/mL) was added to the lipoplex and incubated for 1 h. 100 μL of the IgG^{OX}-(FITC) samples (0.11 mg/mL) were added to the mixture and incubated for 3 h.

Lipoplex 2; liposome^{rhod} and non-labeled siRNA. 30 μL of the liposome^{rhod} stock (3 mg/mL) were pipetted into a plastic eppendorf tube and 23 μL siRNA (0.4 μg/μL) were added slowly to the liposome while vortexing. Then 53 μL PEG²⁰⁰⁰(CHO)₂ (0.135 mg/mL) was added to the lipoplex. 100 μL of the IgG^{OX}-(FITC) samples (0.11 mg/mL) were added to the mixture and incubated for 3 h.

The lipoplexes were purified by an inversed sucrose gradient (20%, 10%, 5% and 0%) in a SORVALL RC M150 GX centrifuge for 1.5 hours at 45000 rpm at 4° C. (FIG. 6d, B). The pink layers were removed from each sample, dialysed in slide-A-Lyzer mini dialysis units (10,000 MWCO, Pierce) and concentrated to 200μL final volume under reduced pressure. The protein concentration in the samples was determined using the BCA assay. 30 μL of each sample (0.11 mg/mL protein) were lyophilized and re-dissolved in 15 μL loading dye. Then the samples were run on a 12% Tris-Glycine SDS page gel (FIG. 6D, A2, lane 3/4). Lipoplexes were also purified by FPLC in a sepharose CL 6B, 20 by 0.6 cm column (0.2 MPa, flow rate 0.4 mL/min, sensitivity 0.05/0.1) A sodium acetate buffer (20 mM sodium acetate and 150 mM NaCi, pH 5.5) was used for the separation and the detector was set at 280 nm. Then the collected fractions were concentrated at the rotavap and the PCS was measured. Typically, two fractions were recovered with the first one giving lipoplex sizes of 200 nm and the second smaller fraction lipoplex sizes of 10,000 nm which are aggregated LsiR-IgG lipoplexes (FIG. 6D, lane 2 and 3 respectively).

(iii) Results

The integrity and activity of the antibody after oxidation was confirmed by SDS page and ELISA, respectively (FIG. 6D). The number of oxidised antibody can be controlled by the incubation time in periodic acid and the concentration of periodic acid. Typically, lh incubation in lOmM periodic acid gives sufficient numbers of oxidized carbohydrates to effectively couple to the CPA lipid in both the liposome and lipoplex without compromising the activity. It is clear from FIG. 6D/A1 that even at increasing incubation times the antibody is unharmed. After coupling of the IgG^{OX} to the lipoplex and FPLC or sucrose gradient purification, the SDS reveals three distinct bands slightly above the 50 kD molecular weight ladder (FIG. 6D, lanes 2/3) which can be attributed to the IgG Fc-fragment with different amounts of CPA lipids coupled. The ELISA of the LsiR-IgG lipoplex demonstrates full activity of the lipoplex coupled antibody.

### Example 7

Cholesteryl Amine 2

Cholesteryl chloroformate 1 (7.5 g, 0.0167 mol) was dissolved in ethylene-1,2-diamine (180 ml) and the mixture stirred for 18 h. The reaction was quenched with water and extracted with dichloromethane. The organic extracts were dried (MgSO₄) and the solvent removed in vacuo to afford a residue which was purified by flash column chromatography [CH₂Cl₂/MeOH/NH₃=192:7:1 followed by CH₂Cl₂/MeOH/NH₃=92:7:1 (v/v)] giving the pure product 2 (5.5 g, 73%) as a white solid (mp 175-177° C.): FTIR (nujol mull) υₘₐₓ[cm⁻¹]3338 (amine), 2977 (alkane), 2830 (alkane), 1692 (carbamate); ¹H NMR (CDCl₃) δ(41) 0.66 (3 H, s, H-18), 0.838-0.854 (3 H, d, H-27 (J=6.4 Hz)), 0.842-0.858 (3 H, d, H-26 (J=6.4 Hz)), 0.890-0.906 (3 H, d, H-21 (J=6.4 Hz)), 0.922 (3 H, s, H-19), 1.02-1.63 (21 H, m, H-1, H-9, H-11, H-12, H-14, H-15, H-16, H-17, H-20, H-22, H-23, H-24, H-25), 1.76-2.1 (5 H, m, H-2, H-7, H-8), 2.22-2.36 (2 H, m, H-4), 2.79-2.81 (2 H, m, H₂NCH₂), 3.197-3.210 (2 H, m, H₂NCH₂CH₂), 4.52 (1 H, m, H-3), 5.31 (1 H, s, H-6); ¹³C NMR (CDCl₃) δ(41) 11.78 (C-18), 18.64 (C-21), 19.26 (C-19), 20.96 (C-11), 22.49 (C-26), 22.75 (C-27), 23.7 (C-23), 24.20 (C-15), 27.92 (C-25), 28.09 (C-2), 28.16 (C-16), 31.77 (C-8), 31.81 (C-7), 35.72 (C-20), 36.09 (C-22) 36.46 (C-10), 36.91 (C-1), 38.50 (C-24), 39.43 (C-4), 39.64 (C-12), 42.2 (C-13), 41.70 (H₂NCH₂CH₂), 43.55 (H₂NCH₂), 49.91 (C-9), 56.04 (C-17), 56.59 (C-14), 74.20 (C-3), 122.39 (C-6), 156.39 (C═O); Mass [ESI/+ve] 473 (M+H); HRMS (FAB/+ve) calc. for C₃₀H₅₃N₂O₂ (M+H) 473.411911; found 473.410704.

### Example 8

Boc-aminoxy Cholesteryl Lipid 3

Boc-amino-oxyacetic acid (145 mg, 0.758 mmol) in anhydrous dichloromethane was treated successively with DMAP (292 mg, 2.39 mmol), HBTU (373 mg, 0.987 mmol) and amine 2 (272 mg, 0.576 mmol) and the mixture stirred at r.t. under a nitrogen atmosphere for 15 h. The reaction was quenched with 7% aqueous citric acid and extracted with dichloromethane. The dried (MgSO₄) extracts were concentrated in vacuo to afford a residue which was purified by flash column chromatography (gradient 20% ethyl acetate/hexane to 65% ethyl acetate/hexane) affording pure Boc-aminoxy cholesteryl lipid 3 (302 mg, 81%). ¹H NMR (400 MHz, CDCl₃) δ(41) 8.56 (s, 1H, BocNHOCH₂), 8.2 (br, CH₂CONHCH₂), 5.5 (m, 1H, chol C6), 5.4 (m, 1 H, chol-O(CO)NH), 4.5 (m, 1H, chol C-3), 4.3 (s, 2H, (CO)CH₂ONH₂), 3.4 (m, 2H, O(CO)NHCH₂CH₂), 3.3 (m, 2H, O(CO)NHCH₂CH₂), 2.32 (m, 2 H, chol C-24), 1.46 (s, 3 H, Boc), 0.94-2.10 (chol C-1, 2, 4, 7, 8, 9, 11, 12, 14, 15, 16, 17, 20, 22, 23, 25), 1.0 (s, 3 H, chol C-19), 0.89 (d, 3 H, J=6.4, chol C-21), 0.83, 0.82 (2×d, 6 H, J=6.5 and 2.0 Hz), 0.68 (s, 3 H, Chol C-18); ¹³C NMR (100 MHz, CDCl₃) δ(41) 169.6 (NH(CO)CH₂ONH₂), 157.9 (Boc), 156.6 (OCONH), 139.7 (C-5), 122.4 (C-6), 82.8 (Boc), 76.2 ((CO)CH₂ONH₂), 74.4 (C-3), 56.6 (C-14), 56.0 (C-17), 49.9 (C-9), 42.2 (C-13), 40.6 (C-4), 39.4-40.6 (C-12, C-4, O(CO)NHCH₂CH₂ overlapping), 38.4 (C-24), 36.9 (C-1), 36.4 (C-10), 36.1 (C-22), 35.7 (C-20), 31.80 (C-8), 321.79 (C-7), 28.1 (C-16 and Boc overlapping), 28.0 (C-2), 27.9 (C-25), 24.2 (C-15), 23.7 (C-23), 22.7 (C-26), 22.5 (C-27), 20.9 (C-11), 19.2 (C-19), 18.6 (C-21) and 11.8 (C-18). Mass [ESI/+ve] 646 [M+H]⁺; HRMS: calc. for C₃₇H₆₄N₃O₆: 646.479512; found: 646.479874.

### Example 9

Cholesteryl Aminoxy Lipid 4

Boc-aminoxy cholesteryl lipid 3 (86 mg, 0.067 mmol) in propan-2-ol (3 ml) was then treated with 4M HCl in dioxane (3 ml) and the mixture stirred at room temperature for 3 h. The solvents were removed in vacuo affording aminoxy lipid 4 (37 mg, 98%); ¹H NMR (400 MHz, d₄-MeOD) δ(41) 5.35 (m, 1H, Chol C6), 4.8 (m, 1 H, chol-O(CO)NH), 4.5 (s, 2H, (CO)CH₂ONH₂), 4.4 (m, 1H, chol C-3), 3.3 (m, 2H, O(CO)NHCH₂CH₂), 3.1 (m, 2H, O(CO)NHCH₂CH₂), 2.32 (m, 2 H, chol C-24), 0.94-2.10 (chol C-1, 2, 4, 7, 8, 9, 11, 12, 14, 15, 16, 17, 20, 22, 23, 25), 1.0 (s, 3 H, chol C-19), 0.89 (d, 3 H, J=6.4, chol C-21), 0.83, 0.82 (2×d, 6 H, J=6.5 and 2.0 Hz), 0.68 (s, 3 H, chol C-18); ¹³C NMR δ[ppm] (100 MHz, CDCl₃) 171.4 (NH(CO)CH₂ONH₂), 158.3 (OCONH), 140.55 (C-5), 123.2 (C-6), 75.4 ((CO)CH₂ONH₂) 71.9 (C-3), 57.5 (C-14), 57.0 (C-17), 51.0 (C-9), 43.0 (C-13), 40.2 (C-4), 40.0-40.6 (C-12, C-4), O(CO)NHCH₂CH₂ overlapping), 39.2 (C-24), 37.8 (C-1), 37.3 (C-10), 36.9 (C-22), 36.6 (C-20), 32.7 (C-8), 32.6 (C-7), 28.9 (C-16), 28.8 (C-2), 28.7 (C-25), 24.9 (C-15), 24.5 (C-23), 23.2 (C-26), 22.9 (C-27), 21.8 (C-11), 19.7 (C-19), 19.2 (C-21) and 12.3 (C-18). Mass [ESI/+ve] 546 [M +H]⁺.

### Example 10

Synthesis of the CPA Compound

The synthesis of cholesteryl—dPEG₄)₂-aminoxy lipid (CPA) 6 was completed in two stages:
1) the solid phase synthesis of the short protected PEG-aminoxy linker 3 (PABoc, Scheme 1) and,
2) the solution phase coupling of cholesteryl-amine (C) to PABoc 3 (Scheme 2).

PABoc 3 was synthesised on 2-Cholorotrityl chloride polystyrene resin [PS-Chlorotrityl-Cl] (Argonaut, USA) using standard peptide Fmoc solid phase methodology. First, short PEG linker, N-Fmoc-amido-dPEG₄™-acid (Quanta BioDesign, Inc., USA) was loaded onto resin under basic conditions and the Fmoc protecting group subsequently removed with piperidine affording amine 1 (Scheme 1). Next another N-Fmoc-amido-dPEG₄™-acid unit was coupled to 1 using HBTU coupling reagent (Novabiochem, UK) and the Fmoc group subsequently deprotected again. The resultant amine was coupled under HBTU conditions to N-Boc-amino-oxyacetic acid (Novabiochem, UK) affording the resin bound PABoc 2 which was then cleaved from the resin under mild acidic condition to afford crude PABoc 3 which was deemed pure enough (TLC) to continue with the next step without further purification.

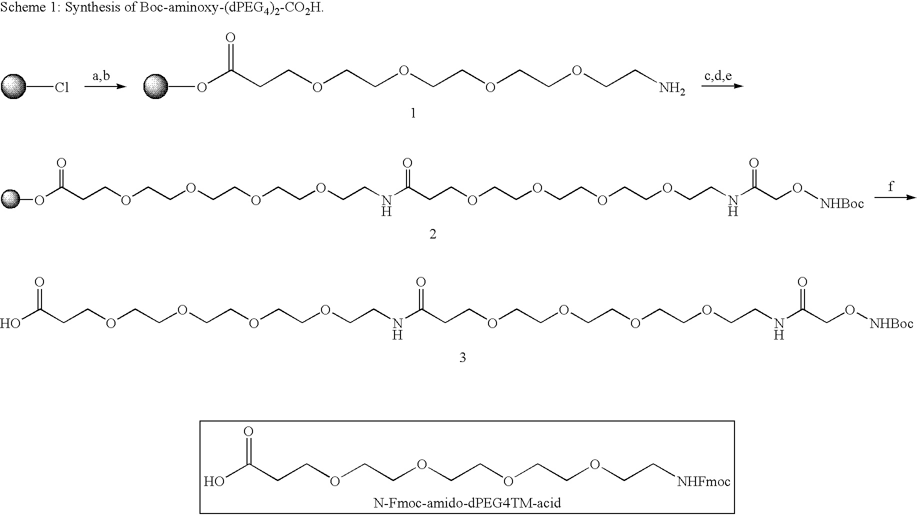

Reagents and conditions: a) N-Fmoc-amido-dPEG₄™-acid (3 equiv.), Hunig base (5 equiv.) in DMF, 2 h., r.t.; b) 20% Piperidine in DMF (3×5 min), r.t.; c) N-Fmoc-amido-dPEG₄™-acid (3 equiv.), HBTU (5 equiv.), Hünig base (5 equiv.) in DMF, 1 h., r.t.; d) 20% Piperidine in DMF (3×5 min), r.t.; e) Boc-amino-oxyacetic acid (3 equiv.), HBTU (5 equiv.), Hünig base (Sequiv.) in DMF, 1 h., r.t.; and f) 50% 1,1,1-trifluoroethanol in DCM, 1 h, r.t.

The completion of the synthesis of CPA is depicted in Scheme 2. Briefly, commercially available cholesteryl chlorofornate (Aldrich, UK) was treated in the neat with excess ethylene diamine generating cholesteryl-amine 4. PABoc 3 was then coupled to amine 4 using HBTU as the coupling reagent affording the Boc-protected cholesteryl-(dPEG₄)₂-arninoxy 3 (71% yield). Deprotection of the Boc-group with 4 M HCl in dioxane yielded the CPA [cholesteryl-(dPEG₄)₂-aminoxy lipid, 6] (>97% yield by analytical HPLC), which was used in biological studies without further purification.

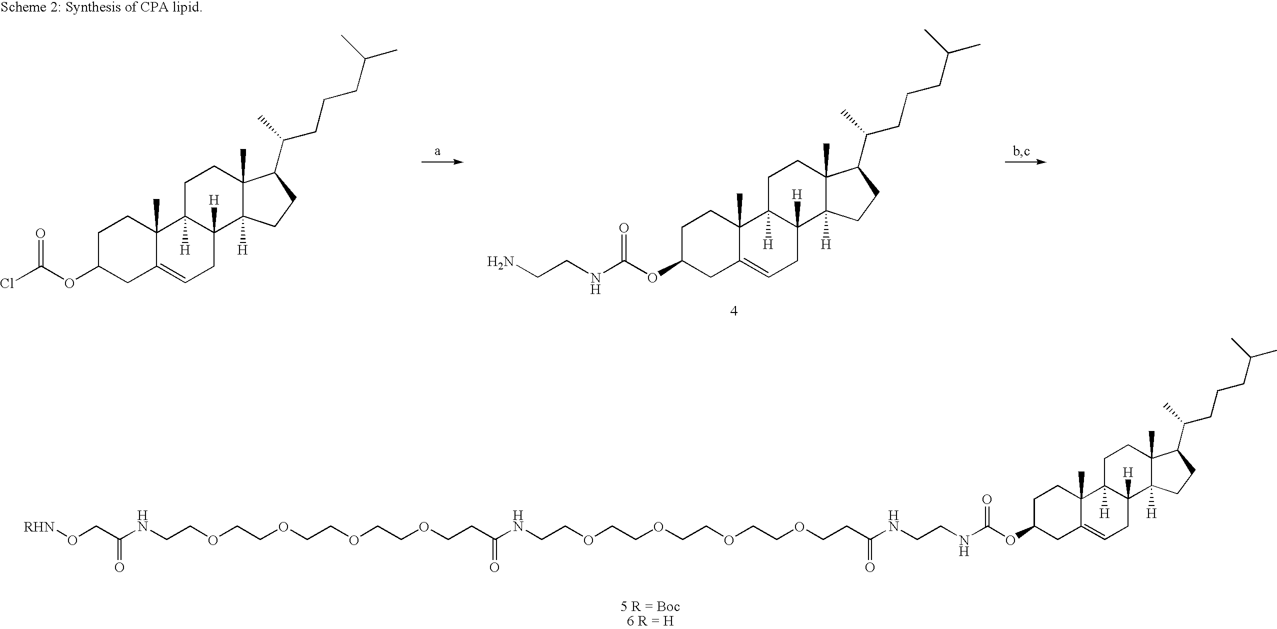

Reagents and conditions: a) ethylene diarnine (large excess), r.t., 18 h, 75%; b) Boc-arnino-oxyacetic acid, HBTU, DMAP, methylene chloride, r.t., 18 h, 81% and c) 4 M HCl/dioxane, propan-2-ol, 3 h, 99%.

Synthesis of Cholesteryl-Gly-PEG (n=11)-Hydrazide (CP₁₁Hyd)

The synthesis of Cholesteryl-Gly-PEG (n=11)-Hydrazide (CP₁₁Hyd) is shown in Scheme 3. Treating cholesterol chloroformate 7 with glycine 8 provides cholesteryl glycine 9 in good yield. Next, O-(2-aminoethyl)-O-[2-(Boc-amino)ethyl]decaethylene glycol is coupled to 9 using the peptide coupling agent HBTU in the presence of DMAP to afford the Boc-protected Cholesteryl-glycine-PEGₙ₌₁₁-amine 10. Removal of the Boc-group with TFA gave free amine 11, which was sufficiently pure to use immediately in coupling N^{h}-tert-butyloxycarbonyl-succinic acid monohydrazide 14, this time using polystyrene-bound DCC-derived resin PS-carbidiimide (Argonaut, UK) as coupling reagent. Boc-protected hydrazide 12 was thus obtained in good 68% yield for the 2 steps. Treatment of 12 with 4 M HCl in dioxane or TFA smoothly afforded the desired hydrazide 13 in 54% yield.

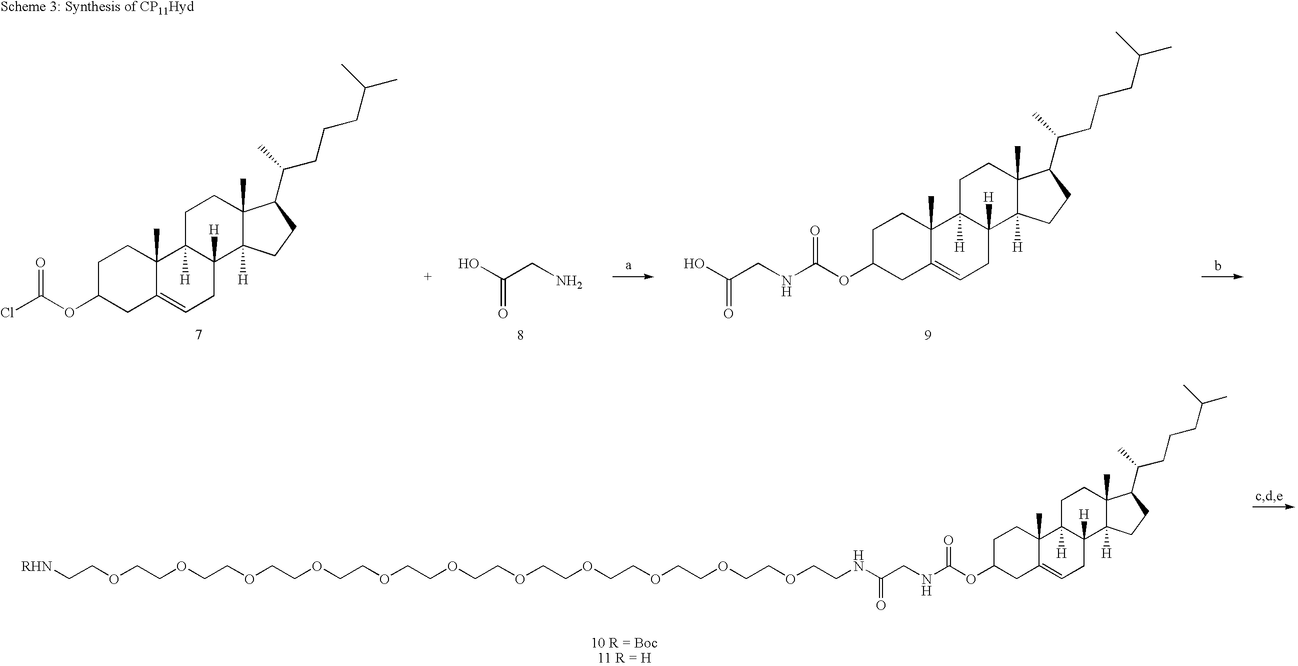

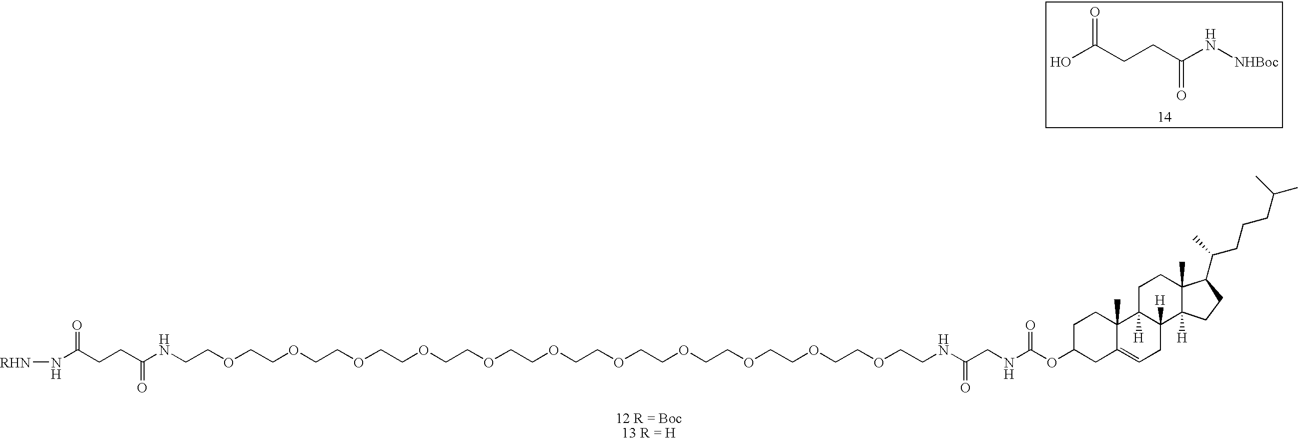

Reagents and conditions: a) Et₃N (1.2 equiv.), dioxane/water, 12 h, r.t., 63%; b) O-(2-aminoethyl)-O-[2-(Boc-amino)ethyl]decaethylene glycol, HBTU, DMAP, DCM, 2 d., r.t., 97%; c) TFA/DCM (1:1), 1 h, r.t.; d) N^{h}-tert-butyloxycarbonyl-succinic acid monohydrazide 14, Et₃N, PS-Carbodiirnide, DCM, 24 h, r.t, 68% for 2 steps; e) 4 M HCl in dioxane, 2-propanol, r.t., 3 h., 54%.

Experimental Procedure

Materials and Methods

Boc-amino-oxyacetic acid and HBTU were obtained from Novabiochem (CN Biosciences, UK). N-Fmoc-amido-dPEG₄™-acid was purchased from Quanta BioDesign Ltd. (Powell, Ohio, USA). PS-Carbodiimide and PS-Chlorotrityl-Cl resins were obtained from Argonaut Technologies, Inc. (Foster City, Calif., USA). All other chemicals were purchased from Sigma Aldrich (Dorset, UK) unless otherwise stated. Dried dichloromethane was distilled with phosphorus pentoxide; other solvents were purchased pre-dried or as required from Sigma-Aldrich (Dorset, UK) or BDH Laboratory Supplies (Poole, UK). HPLC-grade acetonitrile was purchased from Fisher Chemicals (Leicester, UK) and other HPLC-grade solvents from BDH Laboratory Supplies (Poole, UK). Thin layer chromatography (TLC) was performed on pre-coated Merck-Kieselgel 60 F₂₅₄ aluminium backed plated and revealed with ultraviolet light, iodine, acidic ammonium molybdate (IV), acidic ethanolic vanillin, or other agents as appropriate. Flash column chromatography was accomplished on Merck-Kieselgel 60 (230-400 mesh). Mass spectra were recorded using Bruker Esquire 3000, VG-7070B or JEOL SX-102 instruments. ¹H and ¹³C NMR were recorded on Advance Brucker 400 Ultrashield™ machine using residual isotopic solvent as an internal reference (s=singlet, =doublet, t=triplet, q=quartet, quin=quintet, br=broad singlet). Analytical HPLC (Hitachi-LaChrom L-7150 pump system equipped with a Polymer Laboratories PL-ELS 1000 evaporative light scattering detector) was conducted on a Vydac C4 peptide column with gradient 0.1% aqueous TFA to 100% acetonitrile (0.1% TFA) [0-15 min.], then 100% acetonitrile (0.1% TFA) [15-25 min], then 100% methanol [25-45 min].

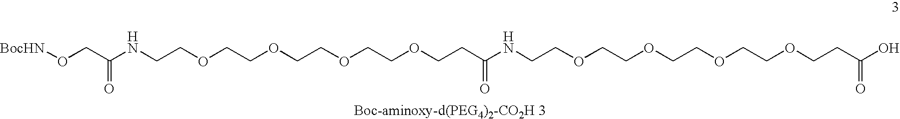

The Boc-aminoxy-dPEG₄)₂-CO₂H 3 was synthesised using a standard peptide solid phase synthesis strategy: Chlorotrityl Chloride resin (1.27 mmol/g loading, 55 mg, 0.070 mmol) was swollen in Dcm for 16 h. The first acid was loaded onto resin by treating the resin with N-Fmoc-amido-dPEG_{4™-acid (}102 mg, 0.209 mmol) and Hünig base (60 μl, 0.349 mmol) in DMF (15 ml) for 1 hour. Fmoc deblocking was achieved by using piperidine (20%) in DMF (2×5 mins) followed by extensive washing with DMF. Next the resultant resin-bound free amine was reacted with N-Fmoc-amido-dPEG_{4™-acid (}102 mg, 0.209 mmol), activated with HBTU (132.5 mg, 0.209 mmol) in Hunig base (60 μl, 0.349 mmol) in DMF (15 ml) for 1 hour. (For each coupling step, 3 equivalent of amino acid, 5 equivalents of DIEA and 3 equivalents of HBTU were used. Each coupling was carried out for 1 hour followed by capping with acetic anhydride (10%) in DMF in the presence of 3 equivalents of DIEA.) Finally, Boc-amino-oxyacetic acid (40 mg) was coupled to yield the resin bound product. The compound was cleaved using 3 mL of a solution consisting of 50% trifluoroethanol in DCM over 4 hours to yield a crude residue (40 mg, 0.058 mmol). ∂_{H} (CDCl₃) 1.48 (9H, Boc), 2.51 (2H, t, J=6.1 Hz, ˜CH2CO2H), 2.59 (2H, t, J=6.05, ˜CH2CONHCH2˜), 3.45 and 3.52 (2H and 2H, m, CONHCH2CH2), 3.55-3.7 (28H, m, CH2OCH2 and CH2OCH2), 3.77 (4H, m, NHCH2CH2O), 4.34 (2H, s, BocHNOCH2CONH), 7.0 (1H, m, BocNHO), 7.9 (1H, m, CH2NHCOCH2) and 8.3 (1H, m, CH2NHCOCH2). ∂c (CDCl₃) 28.2 (Boc), 35.1 (˜CH2CO2H), 36.8 (˜CH2CONHCH2˜), 38.98 and 39.24 (CONHCH2CH2), 66.7 and 67.3 (CH2CH2CO), 69.6 and 69.9 (NHCH2CH2O), 70.3-70.7 (CH2OCH2 and CH2OCH2), 75.8 (BocHNOCH2CONH), 82.5 (quaternary, Boc), 158 (CO, Boc), 169.3 and 171.8 (quaternary, CH2NHCOCH2) and 173.6 (quaternary, CO2H). ESI-MS 684.30 (M−H)⁺.

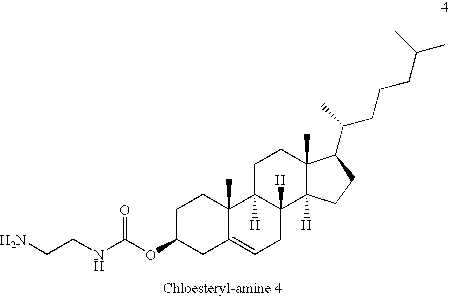

Cholesteryl chlorofornate 1 (7.5 g, 0.0167 mol) was dissolved in ethylene-1,2-diamine (180 ml) and the mixture stirred for 18 h. The reaction was quenched with water and extracted with dichloromethane. The organic extracts were dried (MgSO₄) and the solvent removed in vacuo to afford a residue which was purified by flash column chromatography [CH₂Cl₂: MeOH: NH₃ 192: 7:1→CH₂Cl₂: MeOH: NH₃ 92: 7:1 (v/v)] giving the pure product 2 (5.5 g, 0.0116, 73%) as a white solid (mp 175-177° C.): FTIR (nujol mull) υₘₐₓ 3338 (amine), 2977 (alkane), 2830 (alkane), 1692 (carbamate) cm⁻¹; ¹H NMR (CDCl₃) δ 0.66 (3 H, s, H-18), 0.838-0.854 (3 H, d, H-27 (J=6.4 Hz)), 0.842-0.858 (3 H, d, H-26 (J=6.4 Hz)), 0.890-0.906 (3 H, d, H-21 (J=6.4 Hz)), 0.922 (3 H, s, H-19), 1.02-1.63 (21 H, m, H-1, H-9, H-11, H-12, H-14, H-15, H-16, H-17, H-20, H-22, H-23, H-24, H-25), 1.76-2.1 (5 H, m, H-2, H-7, H-8), 2.22-2.36 (2 H, m, H-4), 2.79-2.81 (2 H, m, H₂NCH₂), 3.197-3.210 (2 H, m, H₂NCH₂CH₂), 4.52 (1 H, m, H-3), 5.31 (1 H, s, H-6); ¹³C NMR (CDCl₃) δ 11.78 (C-18), 18.64 (C-21), 19.26 (C-19), 20.96 (C-11), 22.49 (C-26), 22.75 (C-27), 23.7 (C-23), 24.20 (C-15), 27.92 (C-25), 28.09 (C-2), 28.16 (C-16), 31.77 (C-8), 31.81 (C-7), 35.72 (C-20), 36.09 (C-22) 36.46 (C-10), 36.91 (C-1), 38.50 (C-24), 39.43 (C-4), 39.64 (C-12), 42.2 (C-13), 41.70 (H₂NCH₂CH₂), 43.55 (H₂NCH₂), 49.91 (C-9), 56.04 (C-17), 56.59 (C-14), 74.20 (C-3), 122.39 (C-6), 156.39 (C═O); MS (ESI+ve) 473 (M+H); HRMS (FAB+ve) calcd. for C₃₀H₅₃N₂O₂ (M+H) 473.411911 found 473.410704.

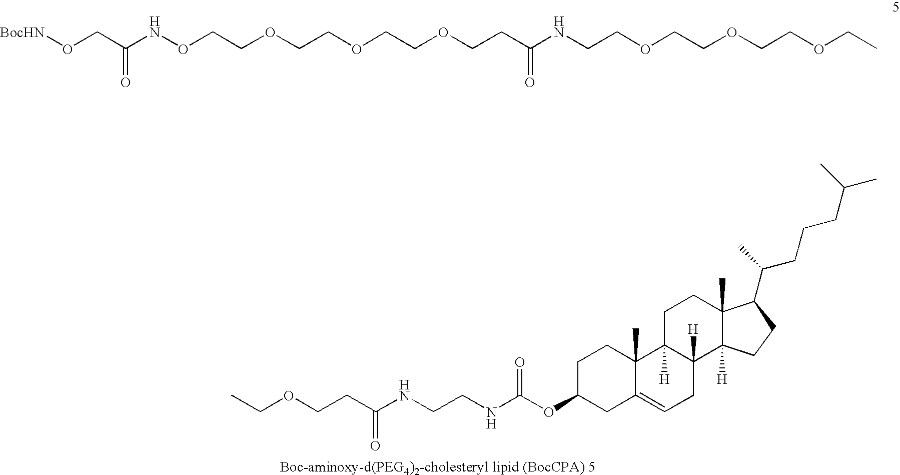

Boc-aminoxy-(dPEG₄)₂-CO₂H (40 mg, 0.058 mmol) in anhydrous dichloromethane was treated successively with DMAP (22 mg, 0.18 mmol), HBTU (24 mg, 0.063 mmol) and cholesteryl amine 2 (28 mg, 0.0.06 mmol) and the mixture stirred at r.t. under a nitrogen atmosphere for 15 h. The reaction was quenched with 7% aqueous citric acid and extracted with dichloromethane. The dried (MgSO₄) extracts were concentrated in vacuo to afford a residue which was purified by flash column chromatography (gradient DCM:MeOH:H₂O) affording pure Boc-aminoxy-dPEG₄)₂-cholesteryl lipid 3 (47 mg, 0.0411 mmol, 71%). ¹H NMR (400 MHz, CDCl₃:MeOD) 5.32 (m, 1H, Chol C6), 4.35 (m, 1H, Chol C-3), 4.28 (s, 2H, (CO)CH₂ONH₂), 3.67 (4H, m, NHCH2CH2O), 3.56-3.61 (24H, m, CH2OCH2 and CH2OCH2), 3.56 (2H, m, CH2CH2CO), 3.50 (2H, m, CH2CH2CO), 3.35 and 3.43 (2H and 2H, m, CONHCH2CH2), 3.24 (m, 2H, CholO(CO)NHCH₂CH₂), 3.18 (m, 2H, CholO(CO)NHCH₂CH₂), 2.42 (4H, m, ˜CH2CO2H and ˜CH2CONHCH2˜), 2.27 (m, 2 H, Chol C-24), 1.46 (s, 3 H, Boc), 0.94-2.10 (Chol C-1, 2, 4, 7, 8, 9, 11, 12, 14, 15, 16, 17, 20, 22, 23, 25), 1.0 (s, 3 H, Chol C-19), 0.89 (d, 3 H, J=6.4, Chol C-21), 0.83, 0.82 (2×d, 6 H, J=6.5 and 2.0 Hz), 0.68 (s, 3 H, Chol C-18); ¹³C NMR (100 MHz, CDCl₃) 173.6 (quaternary, CO2H), 173.3, 172.8 and 170.5 (NH(CO)CH₂ONH₂), 158.5 (Boc), 156.6 (OCONH), 140.166 (C-5), 122.92 (C-6), 82.61 (Boc), 75.77 ((CO)CH₂ONH₂), 74.99 (C-3), 70.4-70.8 (CH2OCH2 and CH2OCH2), 69.81 and 70.04 (NHCH2CH2O), 67.56 and 67.53 (CH2CH2CO), 56.7 (C-14), 56.55 (C-17), 50.5 (C-9), 42.7 (C-13), 40.63 and 39.81 (CholO(CO)NHCH₂CH₂) 40.14 (C-4), 39.88 and 39.58 (CONHCH2CH2), 39.25 (C-12), 38.94 (C-24), 37.3 (C-1), 36.9 (C-10), 36.95 (˜CH2CONHCH2˜), 36.90 (˜CH2CO2H), 36.55 (C-22), 36.17 (C-20), 32.28 (C-8), 32.26 (C-7), 28.5 (C-16 and C-2 overlapping), 28.36 (Boc and C-25), 24.6 (C-15), 24.17 (C-23), 22.99 (C-26), 22.73 (C-27), 21.4 (C-11), 19.6 (C-19), 18.96 (C-21) and 12.11 (C-18). ESI-MS 1162.40 [M+K].

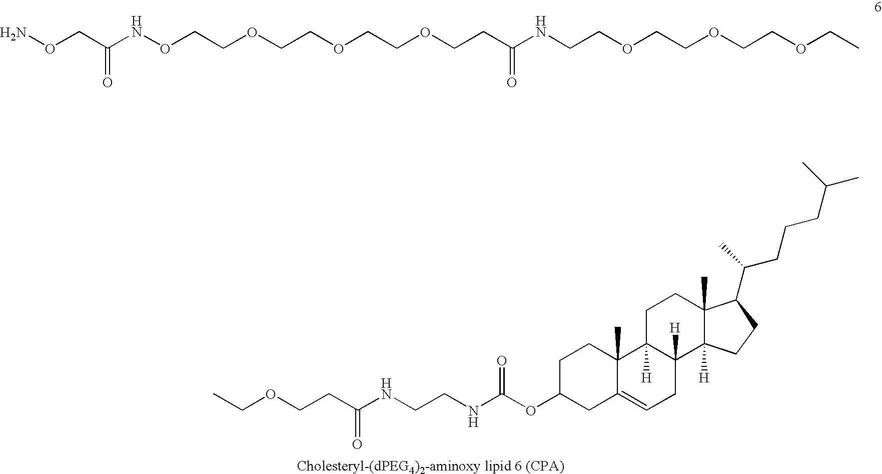

Boc-aminoxy-(dPEG₄)₂-cholesteryl lipid 3 (40 mg, 0.035 mmol) in propan-2-ol (2 ml) was then treated with 4 M HCl in dioxane (2 ml) and the mixture stirred at room temperature for 3 h. The solvents were removed in vacuo affording CPA lipid 4 (37 mg, 98%);. ¹H NMR (400 MHz, d₄-MeOD) 5.31 (m, 1H, Chol C6), 4.57 (s, 2H, (CO)CH₂ONH₂), 4.38 (m, 1H, Chol C-3), 3.69 (4H, m, NHCH2CH2O), 3.53-3.62 (28H, m, CH2OCH2 and CH2OCH2), 3.37 and 3.43 (2H and 2H, m, CONHCH2CH2), 3.26 (m, 2H, CholO(CO)NHCH₂CH₂), 3.19 (m, 2H, CholO(CO)NHCH₂CH₂), 2.45 (4H, m, ˜CH2CO2H and ˜CH2CONHCH2˜), 2.27 (m, 2 H, Chol C-24), 0.94-1.99 (Chol C-1, 2, 4, 7, 8, 9, 11, 12, 14, 15, 16, 17, 20 , 22, 23, 25), 0.97 (s, 3 H, Chol C-19), 0.87 (d, 3 H, J=6.4, Chol C-21), 0.80, 0.82 (2×d, 6 H, J=6.5 and 2.0 Hz), 0.64 (s, 3 H, Chol C-18); ¹³C NMR (100 MHz, CDCl₃) 173.6 (quaternary, CO2H), 173.3, 172.8 and 170.5 (NH(CO)CH₂ONH₂), 157.6 (OCONH), 140.16 (C-5), 122.94 (C-6), 75.03 (C-3), 71.90 ((CO)CH₂ONH₂), 70.4-70.83 (CH2OCH2 and CH2OCH2), 69.54 and 70.14 (NHCH2CH2O), 67.62 (2×CH2CH2CO overlapping), 57.12 (C-14), 56.56 (C-17), 50.50 (C-9), 42.7 (C-13), 40.54 and 39.91 (CholO(CO)NHCH₂CH₂) 40.14 (C-4), 39.88 (C-12), 39.38 and 39.65 (CONHCH2CH2), 38.94 (C-24), 37.3 (C-1), 36.95 (C-10), 36.87 (˜CH2CONHCH2˜), 36.78 (˜CH2CO2H), 36.55 (C-22), 36.17 (C-20), 32.28 (C-8), 32.26 (C-7), 28.5 (C-16 and C-2 overlapping), 28.36 (C-25), 24.6 (C-15), 24.17 (C-23), 22.98 (C-26), 22.73 (C-27), 21.42 (C-11), 19.6 (C-19), 18.96 (C-21) and 12.11 (C-18). ESI-MS 1102.50 [M+K+Na]⁺. Analytical HPLC: 1 peak, RT 31 min.

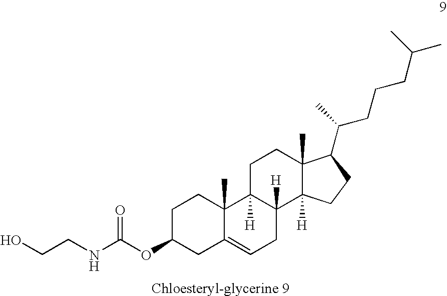

To cholesterolchloroformate 7 (1 g, 2.23 mmol) and triethylamine (424 μl, 2.9 mmol) in dioxane (35 ml) at 0° C. was added glycine 8 dissolved in water (15 ml). The mixture was stirred at room temperature for 12 hours and then the reaction was quenched with 7% aq. Citric acid, the aqueous layer extracted with dichloromethane, and the organic extracts dried (MgSO₄) and concentrated in vacuo. The resultant crude residue was purified by flash column chromatography (EtOAc/Hexanes) to afford the product as a white powder (680 mg, 1.39 mmol, 63%). ¹H NMR (CDCl₃) δ 0.68 (3 H, s, H-18), 0.83-0.87 (3 H, d, H-27 (J=6.4 Hz)), 0.842-0.858 (3 H, d, H-26 (J=6.4 Hz)), 0.890-0.906 (3 H, d, H-21 (J=6.4 Hz)), 0.93 (3 H, s, H-19), 0.99-1.7 (21 H, m, H-1, H-9, H-11, H-12, H-14, H-15, H-16, H-17, H-20, H-22, H-23, H-24, H-25), 1.75-2.2 (5 H, m, H-2, H-7, H-8), 2.2-2.34 (2 H, m, H-4), 3.95 ( br s, CH2˜glycine), 4.5 (1 H, m, H-3), 5.15 (1H, br s, NH), 5.3 (1 H, s, H-6); ¹³C NMR (CDCl₃) δ 11.88 (C-18), 18.74 (C-21), 19.32 (C-19), 21.13 (C-11), 22.55 (C-26), 22.90 (C-27), 23.9 (C-23), 24.40 (C-15), 28.07 (C-25), 28.13 (C-2), 28.3 (C-16), 31.90 (C-8), 31.96 (C-7), 35.88 (C-20), 36.27 (C-22) 36.64 (C-10), 37.0 (H-1), 38.50 (C-24), 39.60 (C-4), 39.83 (C-12), 42.35 (C-13), 42.4, 50.15 (C-9), 56.24 (C-17), 56.80 (C-14), 75.13 (C-3), 122.67 (C-6), 138.8 (C-5), 156.7 (C═O) and 172.2 (COOH).

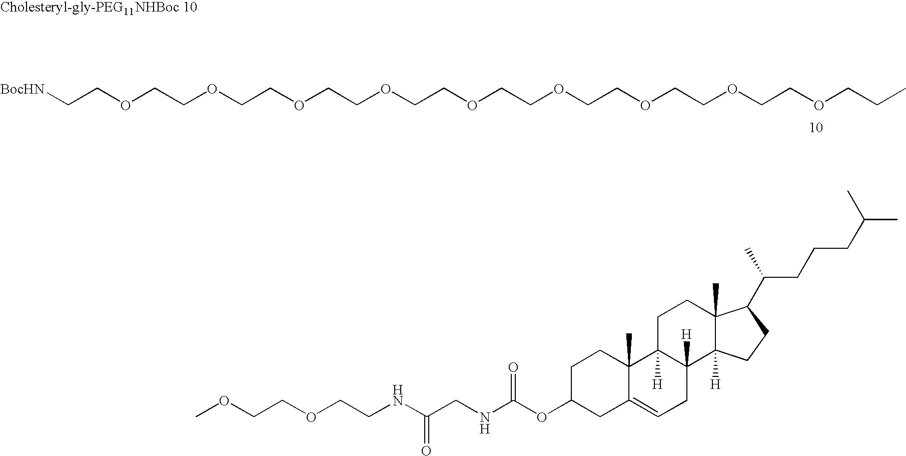

Cholesteryl-glycine 9 (150 mg, 0.309 mmol), O-(2-aminoethyl)-O-[2-(Boc-amino)ethyl]decaethylene glycol (Fluka, UK) (198 mg, 0.307 mmol), HBTU (117 mg, 0.309 mmol) and DMAP (114 mg, 0.927 mmol) were dissolved in anhydrous dichloromethane (50 ml) and stirred under a N2 atmosphere for 2 days. The reaction was quenched with 7% aq. Citric acid, the aqueous layer extracted with dichloromethane/MeOH mixture, and the organic extracts dried (MgSO₄) and concentrated in vacuo. The resultant crude residue was purified by flash column chromatography (CHCL3/MeOH/H2O) to afford the product (335 mg; 0301 mmol, 97%). ¹H NMR (CDCl₃) δ 0.68 (3 H, s, H-18), 0.83-0.87 (3 H, d, H-27 (J=6.4 Hz)), 0.842-0.858 (3 H, d, H-26 (J=6.4 Hz)), 0.890-0.906 (3 H, d, H-21 (J=6.4 Hz)), 0.93 (3 H, s, H-19), 0.99-1.7 (21 H, m, H-1, H-9, H-11, H-12, H-14, H-15, H-16, H-17, H-20, H-22, H-23, H-24, H-25), 1.75-2.2 (5 H, m, H-2, H-7, H-8), 2.2-2.34 (2 H, m, H-4), 3.3-3.7 (48 H, m, PEG), 3.85 (2H, m, CH2˜glycine), 4.5 (1 H, m, H-3), 5.0 (1H, br s, NH), 5.3 (1 H, m, H-6), 5.56 (1H, m, NH) and 6.75 (1H, m, NH); ¹³C NMR (CDCl₃) δ 11.88 (C-18), 18.74 (C-21), 19.36 (C-19), 21.07 (C-11), 22.58 (C-26), 22.84 (C-27), 23.9 (C-23), 24.3 (C-15), 28.03 (C-25), 28.17 (C-2), 28.25 (C-16), 28.46 (Boc), 31.90 (C-8), 31.96 (C-7), 35.88 (C-20), 36.27 (C-22) 36.64 (C-10), 37.0 (H-1), 38.55 (C-24), 39.34 (C-4), 39.54 (C-12), 39.77, 42.35 (C-13), 44.0 (gly), 50.15 (C-9), 56.24 (C-17), 56.74 (C-14), 69.5-70.5 (PEG, 24×CH2), 75.13 (C-3), 80.0 (Boc, quart C), 122.60 (C-6), 139.8 (C-5), 156.7 (C═O), 158 (C═O, Boc) and 170(CONH). HPLC R_{T}=27 min (C-4 column); ES-MS 1136.4 [M +Na]⁺.

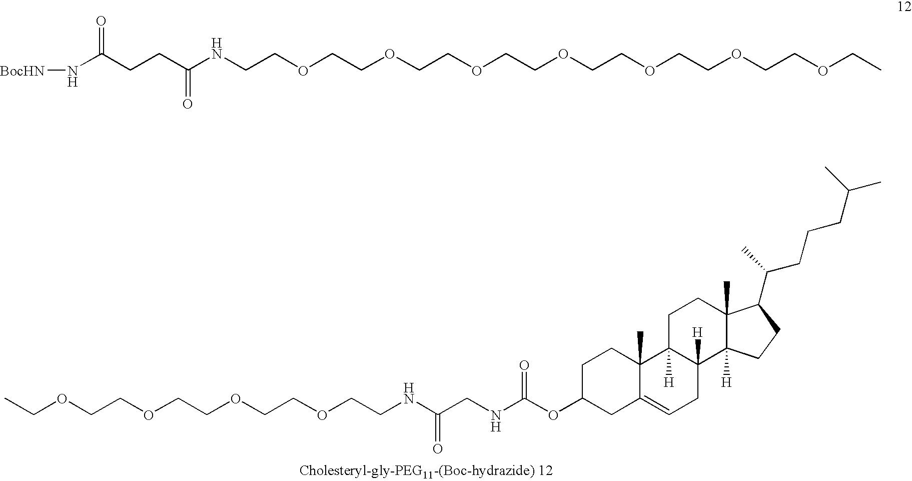

Cholesteryl-gly-PEG₁₁-NHBoc 10 (300 mg) in TFA:DCM (5 ml:5 ml) was stirred at RT for 1 h. The solvent was removed in vacuo affording amine 11 which was used without further purification (see Scheme 3). Thus amine 11 (60 mg, 0.059 mmol), N^{h}-tert-butyloxycarbonyl-succinic acid monohydrazide 14 (232 mg, 0.118 mmol) (synthesized according to Dietzgen et al.; Z. Naturforsch. B; 42, 4, (1987), pp. 441-453) and triethylamine (16 μl) and polystyrene-bound DCC-resin (loading 1.27 mmol/g; 140 mg; 0.1777 mmol), (Argonaut Technologies, UK), were agitated in dichloromethane (10 ml) for 24 h. The resin was removed by filtration and the filtrate concentrated in vacuo affording a residue which was purified by flash column chromatography (chloroform/methanol/water) affording the Boc-protected hydrazide 12 (49 mg, 0.040 mmol, 68%). ¹H NMR (CDCl₃) δ 0.66 (3 H, s, H-18), 0.84-0.86 (3 H, d, H-27 (J=6.4 Hz)), 0.84-0.87 (3 H, d, H-26 (J=6.4 Hz)), 0.89-0.91 (3 H, d, H-21 (J=6.4 Hz)), 0.99 (3 H, s, H-19), 0.99-1.65 (21 H, m, H-1, H-9, H-11, H-12, H-14, H-15, H-16, H-17, H-20, H-22, H-23, H-24, H-25), 1.44 (3H, Boc), 1.75-2.2 (5 H, m, H-2, H-7, H-8), 2.2-2.34 (2 H, m, H-4), 2.55 (4H, m, succinate methylenes), 3.3-3.7 (48 H, m, PEG), 3.85 (2H, m, CH2˜glycine), 4.5 (1 H, m, H-3), 5.35 (1 H, m, H-6), 5.67 (1H, br s, NH), 6.85 (1H, m, NH), 6.94 (1H, m, NH) and 12.13 (1H, br s, NH); ¹³C NMR (CDCl₃) δ 11.8 (C-18), 18.65 (C-21), 19.28 (C-19), 20.99 (C-11), 22.50 (C-26), 22.75 (C-27), 23.77 (C-23), 24.22 (C-15), 27.94 (C-25), 28.08 (C-2), 28.15 (Boc CH3), 28.20 (C-16), 29.70 (succinate CH2), 31.37 (succinate CH2), 31.81 (C-8), 31.81 (C-7), 35.72 (C-20), 36.13 (C-22) 36.51 (C-10), 36.92 (H-1), 38.47 (C-24), 39.25 (C-4), 39.28 (C-12), 39.45, 39.68, 42.26 (C-13), 44.22 (glycine CH2), 45.66 (2×CH2NHCO) 49.97 (C-9), 56.09 (C-17), 56.64 (C-14), 69.5-70.86 (PEG, 24×CH2), 74.74 (C-3), 81.08 (Boc, quart C), 122.50 (C-6), 139.70 (C-5), 155.43 (carbamate C═O), 156.27 (C═O, Boc), 169.4 (glycine CONH), 172.5 (succinate C═O) and 172.23 (succinate C═O). HPLC R_{T}=27 min (C-4 column); ES-MS 1250.3 [M+Na]⁺.

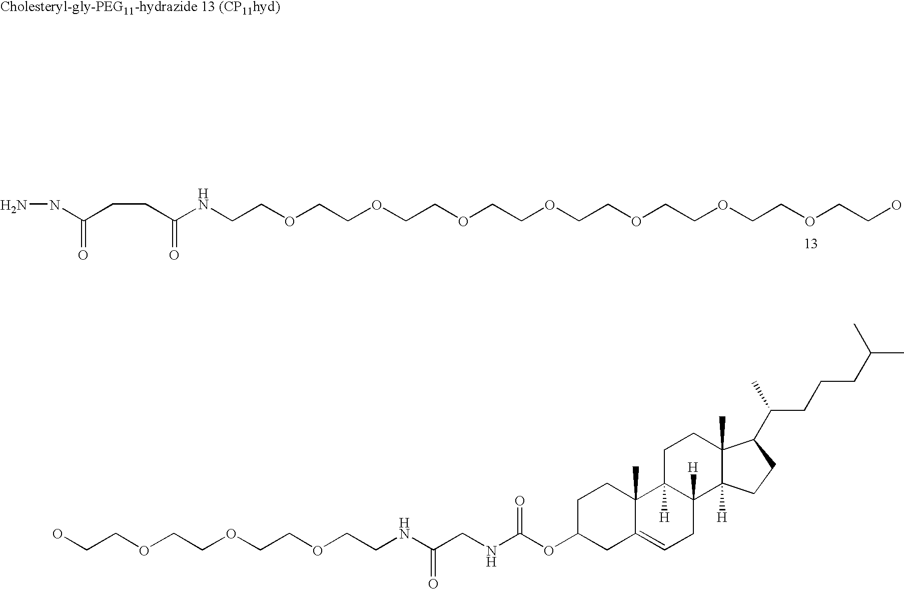

Cholesteryl-gly-PEG₁₁-(Boc-hydrazide) 12 (40 mg, 0.0326 mmol) was dissolved in 2-propanol (3 ml) and then treated with 4 M HCl in dioxane (3 ml). The mixture was stirred for 3 hours and concentrated in vacuo. The residue was purified by ether precipitation for MeOH to afford and off-white gum (20 mg, 0.0177 mmol, 54%). ¹H NMR (CD3OD) δ 0.71 (3 H, s, H-18), 0.84-0.86 (3 H, d, H-27), 0.84-0.87 (3 H, d, H-26 (J=6.4 Hz)), 0.88-0.91 (3 H, d, H-21 (J=6.4 Hz)), 0.99 (3 H, s, H-19), 0.99-1.65 (21 H, m, H-1, H-9, H-11, H-12, H-14, H-15, H-16, H-17, H-20, H-22, H-23, H-24, H-25), 1.75-2.2 (5 H, m, H-2, H-7, H-8), 2.2-2.34 (2 H, m, H-4), 2.6 (4H, m, succinate methylenes), 3.3-3.7 (48 H, m, PEG), 3.85 (2H, m, CH2˜glycine), 4.45 (1 H, m, H-3) and 5.35 (1 H, m, H-6); ¹³C NMR (CD3OD) δ 12.6 (C-18), 18.65 (C-21), 19.28 (C-19), 20.99 (C-11), 22.50 (C-26), 22.75 (C-27), 23.77 (C-23), 24.22 (C-15), 27.94 (C-25), 28.08 (C-2), 28.15 (Boc CH3), 28.20 (C-16), 29.70 (succinate CH2), 31.37 (succinate CH2), 31.81 (C-8), 31.81 (C-7), 35.72 (C-20), 36.13 (C-22) 36.51 (C-10), 36.92 (H-1), 38.47 (C-24), 39.25 (C-4), 39.28 (C-12), 39.45, 39.68, 42.26 (C-13), 44.22 (glycine CH2), 45.66 (2×CH2NHCO) 49.97 (C-9), 56.09 (C-17), 56.64 (C-14), 69.5-70.86 (PEG, 24×CH2), 74.74 (C-3), 81.08 (Boc, quart C), 122.50 (C-6), 139.70 (C-5), 155.43 (carbamate C═O), 156.27 (C═O, Boc), 169.4 (glycine CONH), 172.5 (succinate C═O) and 172.23 (succinate C═O). HPLC R_{T}=27 min (C-4 column); ES-MS 1150.3 [M+Na]⁺

### Example 11

Freeze Dried and Rehydrated LsiR Lipoplexes

Multilamellar liposomes were prepared in deionised water by hydrating a lyophilized powder of CDAN/DOPE (50:50, m/m) at 3 mg/mL lipid. The siRNA was diluted in water and slowly added to the liposomes while vortexing to give the LsiR complexes at a final siRNA concentration of 20 μg/mL (siRNA) at a lipid/siRNA ratio 12:1 (w/w). Sucrose, trehalose and lactose (Sigma, UK) stock solutions of 30% (w/v) were prepared and appropriate volumes of these added to the LsiR lipoplexes to result in final concentrations of 5, 10 and 20% (w/v), respectively. The LsiR concentration at the complex formation varied depending on the amount of cryo-protectant used. For example, the LsiRs without cryo-protectant were prepared at 20 μg/mL but the LsiRs containing 20% cryo-protectant were prepared at 60 μg/mL. 25 μL of lipoplexes (0.5 μg siRNA) were freeze-dried and the resulting powder hydrated in water at 20 μg/mL (25 μL) or at 5 μg/mL (100 μL), vortexed and allowed to stand at room temperature for 15 min. Size measurement of these LsiR particles by PCS demonstrated that the particles were exhibiting identical sizes as prior to the lyophilization/rehydration process.

pDNA transfection was carried out using 0.2 μg/well pDNA/well as described. 0.1 μg/well of these freeze dried/rehydrated LsiR was incubated for 3 h at 37° C./10% CO₂ 3 h post-pDNA transfection. In brief, for fresh LsiR and freeze-dried/rehydrated LsiR at 20 μg/mL (siRNA), 5 μL of the respective complexes were added to 250 μL of complete growth medium in each well of growing cells. For the LsiR thawed/rehydrated at 5 μg/mL (siRNA), 20 L of complex was added to 250 μL of complete growth medium in each well of growing cells. After 3 h incubation, the siFECTion medium was removed and replaced with 400 μL of fresh complete growth medium.

The results are shown in FIG. 8.

Surprisingly, the LsiR lipoplexes freeze-dried in the presence of trehalose and rehydrated in either 25 or 100 μl water are even better in down-regulating the lacZ reporter gene than the freshly prepared LsiR lipoplex.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology and molecular biology or related fields are intended to be within the scope of the following claims.

### REFERENCES

1. Keller, M., Wohr, T., Dumy, P., Patiny, L., and Mutter, M. (2000) Chem.-Eur. J 6, 4358-4363
2. Zhang, Y. P., Sekirov, L., Saravolac, E. G., Wheeler, J. J., Tardi, P., Clow, K., Leng, E., Sun, R., Cullis, P. R., and Scherrer, P. (1999) Gene Ther. 6, 1438-1447
3. Reddy, J. A., and Low, P. S. (2000) J. Control. Release 64, 27-37
4. Erbacher, P., Bettinger, T., Belguise-Valladier, P., Zou, S. M., Coll, J. L., Behr, J. P., and Remy, J. S. (1999) J. Gene. Med. 1, 210-222
5. Lasic, D. D., Vallner, J. J., and Working, P. K. (1999) Curr. Opin. Mol. Ther. 1, 177-185
6. Iden, D. L., and Allen, T. M. (2001) Biochim. Biophys. Acta-Biomembr. 1513, 207-216
7. Lasic, D. D., and Papahadjopoulos, D. (1995) Science 267, 1275-1276
8. Lasic, D. D. (1997) J. Control. Release 48, 203-222
9. Keller, M., Harbottle, R. P., Perouzel, E., Morvane, C., Imran, S., Ahad, R., Vaysse, L., Bergau, A., Moritz, S., Brahimi-Hom, M. C., Coutelle, C., and Miller, A. D. (2003) ChemBioChem 4, 286-298
10. Song, L. Y., Ahkong, Q. F., Rong, Q., Wang, Z., Ansell, S., Hope, M. J., and Mui, B. (2002) Biochim. Biophys. Acta-Biomembr. 1558, 1-13
11. Shi, F. X., Wasungu, L., Nomden, A., Stuart, M. C. A., Polushkin, E., Engberts, J., and Hoekstra, D. (2002) Biochem. J. 366, 333-341
12. Tang, G. L., Reinhart, B. J., Bartel, D. P., and Zamore, P. D. (2003) Genes Dev. 17, 49-63
13. Catalanotto, C., Azzalin, G., Macino, G., and Cogoni, C. (2002) Genes Dev. 16, 790-795
14. Gitlin, L., Karelsky, S., and Andino, R. (2002) Nature 418, 430-434
15. Guo, S., and Kemphues, K. J. (1995) Cell 81, 611-620
16. Fire, A., Xu, S. Q., Montgomery, M. K., Kostas, S. A., Driver, S. E., and Mello, C. C. (1998) Nature 391, 806-811
17. Elbashir, S. M., Harborth, J., Lendeckel, W., Yalcin, A., Weber, K., and Tuschl, T. (2001) Nature 411, 494-498
18. Capodici, J., Kariko, K., and Weissman, D. (2002) J. Immunol. 169, 5196-5201
19. Martinez, J., Patkaniowska, A., Urlaub, H., Luhrmann, R., and Tuschl, T. (2002) Cell 110, 563-574
20. Abdelrahim, M., Samudio, I., Smith, R., Burghardt, R., and Safe, S. (2002) J. Biol. Chem. 277, 28815-28822
21. Hasuwa, H., Kaseda, K., Einarsdottir, T., and Okabe, M. (2002) FEBS Lett. 532, 227-230
22. Sorensen, D. R., Leirdal, M., and Sioud, M. (2003) J. Mol. Biol. 327, 761-766
23. Sledz, C. A., Holko, M., de Veer, M. J., Silverman, R. H., and Williams, B. R. G. (2003) Nat. Cell Biol. 5, 834-839
24. Bridge, A. J., Pebemard, S., Ducraux, A., Nicoulaz, A. L., and Iggo, R. (2003) Nature Genet. 34, 263-264
25. Feigner, P. L., Gadek, T. R., Holm, M., Roman, R., Chan, H. W., Wenz, M., Northrop, J. P., Ringold, G. M., and Danielsen, M. (1987) Proc. Natl. Acad. Sci. USA 84, 7413-7417
26. Zelphati, O., Nguyen, C., Ferrari, M., Felgner, J., Tsai, Y., and Felgner, P. L. (1998) Gene Ther. 5, 1272-1282
27. Hansma, H. G., Golan, R., Hsieh, W., Lollo, C. P., MullenLey, P., and Kwoh, D. (1998) Nucleic Acids Res. 26, 2481-2487
28. Golan, R., Pietrasanta, L. I., Hsieh, W., and Hansma, H. G. (1999) Biochemistry 38, 14069-14076
29. Bloomfield, V. A. (1996) Curr. Op. Struct. Biol. 6, 334-341
30. Boulikas, T., and Martin, F. (1997) Int. J. Onc. 10, 317-322
31. Keller, M., Tagawa, T., Preuss, M., and Miller, A. D. (2002) Biochemistry 41, 652-659
32. Bloomfield, V. A. (1991) Biopolymers 31, 1471-1481
33. Kreiss, P., Cameron, B., Rangara, R., Mailhe, P., Aguerre-Charriol, O., Airiau, M., Scherman, D., Crouzet, J., and Pitard, B. (1999) Nucleic Acids Res. 27, 3792-3798
34. Chesnoy, S., and Huang, L. (1999) Stp. Pharm. Sci. 9, 5-12
35. Ross, P. C., and Hui, S. W. (1999) Gene Ther. 6, 651-659
36. Simberg, D., Danino, D., Talmon, Y., Minsky, A., Ferrari, M. E., Wheeler, C. J., and Barenholz, Y. (2001) J. Biol. Chem. 276, 47453-47459
37. Zuidam, N. J., HirschLemer, D., Margulies, S., and Barenholz, Y. (1999) Biochim. Biophys. Acta-Biomembr. 1419, 207-220
38. Zuidam, N. J., and Barenholz, Y. (1999) Int. J. Pharm. 183, 43-46
39. Stewart, L., Manvell, M., Hillery, E., Etheridge, C. J., Cooper, R. G., Stark, H., van-Heel, M., Preuss, M., Alton, E., and Miller, A. D. (2001) J. Chem. Soc.-Perkin Trans. 2, 624-632
40. Keller, M., Jorgensen, M. R., Perouzel, E., and Miller, A. D. (2003) Biochemistry in press
41. Brigham, K. L., Meyrick, B., Christman, B., Conary, J. T., King, G., Berry, L. C., and Magnuson, M. A. (1993) American Journal of Respiratory Cell and Molecular Biology 8, 209-213
42. Torchilin, V. P., Omelyanenko, V. G., Papisov, M. I., Bogdanov, A. A., Trubetskoy, V. S., Herron, J. N., Gentry, C. A. Biochimica Et Biophysica Acta-Biomembranes 1195, 11-20 (1994).

1. A delivery vector comprising a liposome, wherein one or more lipids of the liposome are coupled, reversibly or irreversibly, to one or more polymers, and wherein the liposome comprises siRNA.

2. A delivery vector according to claim 1, wherein the one or more lipids of the liposome that are coupled, reversibly or irreversibly, to one or more polymers, are exposed at the surface of the liposome.

3. A delivery vector according to claim 1, wherein the liposome comprises one or more aminoxy group containing lipids of the formula (I): 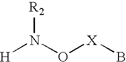
wherein B is a lipid; wherein X is an optional linker group and wherein R₂ is H or a hydrocarbyl group.

4. A delivery vector according to claim 3, wherein the aminoxy group containing lipid is CPA.

5. delivery vector according to claim 1, wherein the liposome comprises one or more cationic lipids and/or one or more non-cationic co-lipids.

6. A delivery vector according to claim 5, wherein the cationic lipid comprises at least one alicyclic group.

7. A delivery vector according to claim 6, wherein the at least one alicyclic group is cholesterol.

8. A delivery vector according to claim 6, wherein the cationic lipid is N¹-cholesteryloxycarbonyl-3,7-diazanononane-1,9-diamine (CDAN).

9. A delivery vector according to claim 5, wherein the non-cationic co-lipid is a phosphatidylethanolamine.

10. A delivery vector according to claim 9, wherein the non-cationic co-lipid is dioleoyl phosphatidylethanolamine (DOPE).

11. A delivery vector according to claim 1, wherein the polymer comprises one or more aldehyde and/or ketone groups.

12. A delivery vector according to claim 1, wherein the polymer is PEG.

13. A delivery vector according to claim 12, wherein the liposome is coupled with from about 0.1 to about 5% PEG.

14. A delivery vector according to claim 1, wherein the liposome comprises or is coupled, reversibly or irreversibly to, one or more agents.

15. A targeted delivery vector comprising a liposome, wherein one or more lipids of the liposome are coupled, reversibly or irreversibly, to one or more polymers and one or more agents, and wherein the liposome comprises siRNA.

16. A targeted delivery vector according to claim 15, wherein the agent(s) is selected from the group consisting of sugar, carbohydrate and a ligand.

17. A targeted delivery vector according claim 16, wherein the sugar is selected from the group consisting of glucose, mannose, lactose, fructose, maltotriose, maltoheptose.

18. A targeted delivery vector according to claim 16, wherein the ligand is an antibody.

19. A method for delivering siRNA, comprising the step of providing to the environment of a cell, tissue or organ the delivery vector according to claim 1.

20. A delivery vector according to claim 18 for use in the delivery of siRNA to a cell, tissue or organ.

21. Use of a delivery vector according to claim 1 in the manufacture of a composition for the delivery of siRNA to a cell, tissue or organ.

22. A process for preparing a delivery vector comprising a liposome, wherein one or more lipids of the liposome are coupled, reversibly or irreversibly, to one or more polymers, and wherein the liposome comprises siRNA, comprising the steps of:
(i) contacting the siRNA with a liposome; and
(ii) coupling, reversibly or irreversibly, the liposome formed in step (i) to the polymer(s).

23. A process according to claim 22, comprising the additional step of:
(iii) coupling, reversibly or irreversibly, the liposome formed in step (i) or step (ii) with one or more agent(s).

24. A process for preparing a targeted delivery vector comprising a liposome, wherein one or more lipids of the liposome are coupled, reversibly or irreversibly, to one or more polymers and one or more agents, and wherein the liposome comprises siRNA, comprising the steps of:
(i) contacting the siRNA with the liposome;
(ii) coupling, reversibly or irreversibly, the liposome formed in step (i) to a polymer(s); and
(iv) coupling, reversibly or irreversibly, the liposome formed in step (i) or step (ii) with one or more agent(s).

25. A method comprising the steps of:
(i) providing a vector according to claim 1;
(ii) optionally contacting the vector with a cryo-protectant; and
(iii) freeze-drying the vector.

26. A method according to claim 25, wherein the cryo-protectant is selected from the group consisting of sucrose, trehalose and lactose.

27. A method according to claim 25, comprising the additional step of: (iv) rehydrating the vector prior to use.

28. A freeze-dried vector obtainable or obtained by the method of claim 25.

29. A liposome comprising a lipid and a coupling moiety wherein the distance between the lipid and the coupling moiety is at least 1.5 nm.

30. A liposome according to claim 29 of the formula 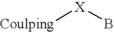
wherein B is a lipid; wherein X is a linker group and Coupling is a coupling moiety, wherein the X backbone comprises at least 30 atoms.

31. A liposome according to claim 30 wherein the X backbone comprises at least 40 atoms

32. A liposome according to claim 30 wherein X is or comprises a group of the formula 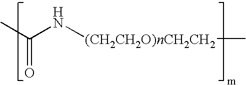
wherein n and m are independently from 0 to 6, preferably from 1 to 6, more preferably 2, 3 or 4, more preferably 2 or 4.

33. A liposome according to claim 30 wherein X is or comprises a group of the formula 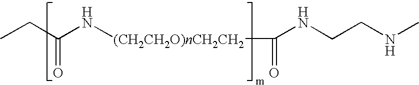
wherein n and m are independently from 0 to 6, preferably from 1 to 6, more preferably 2, 3 or 4, more preferably 2 or 4.

34. A liposome according to claim 30 wherein X is or comprises a group of the formula 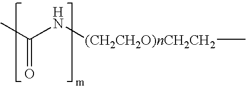
wherein m is from 0 to 6, preferably from 1 to 6, more preferably 1, 2 or 3, more preferably 1 and wherein n is from 0 to 20, preferably from 5 to 15, more preferably 10, 11 or 12, more preferably 11.

35. A liposome according to claim 30 wherein X is or comprises a group of the formula 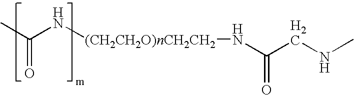
wherein m is from 0 to 6, preferably from 1 to 6, more preferably 1, 2 or 3, more preferably 1 and wherein n is from 0 to 20, preferably from 5 to 15, more preferably 10, 11 or 12, more preferably 11.

36. A pharmaceutical composition comprising the delivery vector according to claim 1 a pharmaceutically acceptable carrier or diluent.

37. A method of treating a disease in a subject comprising administering to said subject a medically effective amount of a delivery vector according to claim 1.

38. A delivery vector according to claim 1 for use in the treatment of a disease.

39. Use of a delivery vector according to claim 1 in the manufacture of a composition for the treatment of a disease.

40. A method according to claim 37, wherein the disease is liver disease and/or liver damage.

41. Use of a liposome coupled to a polymer in the preparation of a delivery vector comprising siRNA.

42. Use of a liposome coupled to a polymer and one or more agents in the preparation of a targeted delivery vector comprising siRNA.

43. (canceled)

44. (canceled)

45. (canceled)

46. (canceled)
